(19) United States
(12) Patent Application Publication
(10) Pub. No.: US 2005/0170528 A1
(43) Pub. Date: Aug. 4, 2005

(54) Binary prediction tree modeling with many predictors and its uses in clinical and genomic applications

(76) Inventors: Mike West, Durham, NC (US); Joseph R. Nevins, Chapel Hill, NC (US)

Correspondence Address: ROPES & GRAY LLP, ONE INTERNATIONAL PLACE, BOSTON, MA, 02110-2624, US

(21) Appl. No.: 10/692,002

(22) Filed: Oct. 24, 2003

Related U.S. Application Data
(60) Provisional application No. 60/458,373, filed on Mar. 31, 2003.
(60) Provisional application No. 60/457,887, filed on Mar. 26, 2003.
(60) Provisional application No. 60/448,461, filed on Feb. 21, 2003.
(60) Provisional application No. 60/448,462, filed on Feb. 21, 2003.
(60) Provisional application No. 60/425,256, filed on Nov. 12, 2002.
(60) Provisional application No. 60/424,701, filed on Nov. 8, 2002.
(60) Provisional application No. 60/420,729, filed on Oct. 24, 2002.
(60) Provisional application No. 60/421,102, filed on Oct. 25, 2002.
(60) Provisional application No. 60/421,062, filed on Oct. 25, 2002.
(60) Provisional application No. 60/424,718, filed on Nov. 8, 2002.
(60) Provisional application No. 60/424,715, filed on Nov. 8, 2002.

(51) Int. Cl. G06F 19/00; G01N 33/48; G01N 33/50; G01N 33/543

(52) U.S. Cl. 436/518; 702/19

(57) ABSTRACT

The statistical analysis described and claimed is a predictive statistical tree model that overcomes several problems observed in prior statistical models and regression analyses, while ensuring greater accuracy and predictive capabilities. Although the claimed use of the predictive statistical tree model described herein is directed to the prediction of a disease in individuals, the claimed model can be used for a variety of applications including the prediction of disease states, susceptibility of disease states or any other biological state of interest, as well as other applicable non-biological states of interest. This model first screens genes to reduce noise, applies k-means correlation-based clustering targeting a large number of clusters, and then uses singular value decompositions (SVD) to extract the single dominant factor (principal component) from each cluster. This generates a statistically significant number of cluster-derived singular factors, that we refer to as metagenes, that characterize multiple patterns of expression of the genes across samples. The strategy aims to extract multiple such patterns while reducing dimension and smoothing out gene-specific noise through the aggregation within clusters. Formal predictive analysis then uses these metagenes in a Bayesian classification tree analysis. This generates multiple recursive partitions of the sample into subgroups (the “leaves” of the classification tree), and associates Bayesian predictive probabilities of outcomes with each subgroup. Overall predictions for an individual sample are then generated by averaging predictions, with appropriate weights, across many such tree models. The model includes the use of iterative out-of-sample, cross-validation predictions leaving each sample out of the data set one at a time, refitting the model from the remaining samples and using it to predict the hold-out case. This rigorously tests the predictive value of a model and mirrors the real-world prognostic context where prediction of new cases as they arise is the major goal.

### FIELD OF THE INVENTION

The field of this invention is the application of classification tree models incorporating Bayesian analysis to the statistical prediction of binary outcomes especially in clinical, genomic and medical applications.

### BACKGROUND OF THE INVENTION

Bayesian analysis is an approach to statistical analysis that is based on the Bayes's law, which states that the posterior probability of a parameter p is proportional to the prior probability of parameter p multiplied by the likelihood of p derived from the data collected. This increasingly popular methodology represents an alternative to the traditional (or frequentist probability) approach: whereas the latter attempts to establish confidence intervals around parameters, and/or falsify a-priori null-hypotheses, the Bayesian approach attempts to keep track of how a-priori expectations about some phenomenon of interest can be refined, and how observed data can be integrated with such a-priori beliefs, to arrive at updated posterior expectations about the phenomenon.

Bayesian analysis have been applied to numerous statistical models to predict outcomes of events based on available data. These include standard regression models, e.g. binary regression models, as well as to more complex models that are applicable to multi-variate and essentially non-linear data. Another such model is commonly known as the tree model which is essentially based on a decision tree.

Decision trees can be used in clarification, prediction and regression. A decision tree model is built starting with a root mode, and training data partitioned to what are essentially the “children” modes using a splitting rule. For instance, for clarification, training data contains sample vectors that have one or more measurement variables and one variable that determines that class of the sample.

Various splitting rules have been used; however, the success of the predictive ability varies considerably as data sets become larger. Furthermore, past attempts at determining the best splitting for each mode is often based on a “purity” function calculated from the data, where the data is considered pure when it contains data samples only from one clan. Most frequently used purity functions are entropy, gini-index, and towing rule. The success of each of these tree models varies considerably and their applicability to complex biological and molecular data is often prone to difficulties. Thus, there is a med statistical model that can consistently deliver accurate results with high predictive capabilities. The present invention describes a statistical predictive tree model to which Bayesian analysis is applied incorporating several key innovations described herewith.

The statistical analysis enabled by the statistical models of the present invention enable a predictive analysis of complex multi-variable data to predict an outcome of a state. Such outcomes include, but are not limited to, biological outcomes, such as clinical and medical outcomes. In a preferred embodiment, such cliniical and/or medical outcomes are the occurrence of a disease or a disease state based on the statistical analysis of clinical and/or genomic data. The present invention allows the integration of currently accepted risk factors with genomic data and carries the promise of focusing the practice of medicine on the individual patient—not merely to groups of patient populations. Such integration requires interpreting the complex, multivariate patterns in gene expression data, and evaluating their capacity to improve clinical predictions. The present invention enables this in a study of predicting nodal metastatic states and relapse for breast cancer patients.

The present invention identifies aggregate patterns of gene expression termed metagenes that associate with disease state indicators such as lymph node status and with recurrence, and that are capable of honestly predicting outcomes in individual patients with about 90% accuracy. The identified metagenes define distinct groups of genes, suggesting different biological processes underlying these two characteristics of breast cancer. This is important from both a regulatory, mechanistic and clinical perspective.

Multiple aggregate measures of gene expression profiles define valuable predictive associations with clinical indicators for the individual patient. These results indicate the potential for gene expression data to aid in achieving more accurate individualized prognosis. Importantly, this is evaluated in terms of precise numerical predictions, via ranges of probabilities of outcome, for the individual patient. Such precise and statistically valid assessments of patient-specific risk will ultimately be of most value to clinical practitioners faced with treatment decisions.

Genomic information, in the form of gene expression signatures, has an established capacity to define clinically relevant risk factors in disease prognosis. Recent studies have generated such signatures related to lymph node metastasis and disease recurrence in breast cancer (See West, M. et al. Predicting the clinical status of human breast cancer by using gene expression profiles. Proc. Natl. Acad. Sci., USA 98, 11462-11467 (2001); Spang, R. et al. Prediction and uncertainty in the analysis of gene expression profiles. In Silico Biol. 2, 0033 (2002); van'T Veer, L. J. et al. Gene expression profiling predicts clinical outcome of breast cancer. Nature 415, 530-536 (2002); van de Vijver, M. J. et al. A gene-expression signature as a predictor of survival in breast cancer. N. Engl. J. Med. 347, 1999-2009 (2002); Huang, E. et al. Gene expression predictors of breast cancer outcomes. Lancet in press, (2003)) as well as in other cancers (See Pomeroy, S. L. et al. Prediction of central nervous system embryonal tumour outcome based on gene expression. Nature 415, 436-442 (2002); Alizadeh, A. A. et al. Distinct types of diffuse large B-cell lymphoma identified by gene expression profiling. Nature 403, 503-511 (2000); Rosenwald, A. et al. The use of molecular profiling to predict survival after chemotherapy for diffuse large-B-cell lymphoma; Bhattacharjee, A. et al. Classification of human lung carcinomas by mRNA expression profiling reveals distinct adenocarcinoma subclasses. Proc. Natl. Acad. Sci. USA 98, 13790-13795 (2001); Ramaswamy, S. et al. Multiclass cancer diagnosis using tumor gene expression signatures. Proc. Nat'l. Acad. Sci. 98, 15149-15154 (2001); Golub, T. R. et al. Molecular classification of cancer: class discovery and class prediction by gene expression monitoring. Science 286, 531-537 (1999); Shipp, M. A. et al. Diffuse large B-cell lymphoma outcome prediction by gene expression profiling and supervised machine learning. Nat. Med. 8, 68-74 (2002); Yeoh, E.-J. et al. Classification, subtype discovery, and prediction of outcome in pediatric acute lymphoblastic leukemia by gene expression profiling. Cancer Cell 1, 133-143 (2002)) and non-cancer disease contexts. The challenge addressed by the instant invention is the integration of such genomic information into prognostic models that can be applied in a clinical setting to improve the accuracy of treatment decisions as well as the development of new treatment and drug regiments for the treatment of disease.

Two issues are critical in achieving this goal. First, we need modeling approaches that focus on the generation of predictions for the individual patient rather than associating risks for large groups of patients are required. Second, we statistical models that can discover and evaluate interactions of multiple risk factors, and combine them to produce informed predictions are needed. Although gene expression profiles may prove to be more powerful indicators of tumor behavior, analysis should not force a choice of one form of data over the other; all forms of data should be accommodated and evaluated. As new technologies develop, new forms of genomic data will be capable of improving prediction of disease outcomes; analytic models must therefore be technology-independent and able to accommodate emerging forms of molecular and clinical data. This integrative view underlies the development of clinico-genomic models in the instant invention. Thus, it permits a more integrative approach to prognostic systems in support of personalized health planning.

### SUMMARY OF THE INVENTION

This invention discusses the generation and exploration of classification tree models, with particular interest in problems involving many predictors. Problems involving multiple predictors arise in situations where the prediction of an outcome is dependent on the interaction of numerous factors (predictors), such as the prediction of clinical or physiological states using various forms of molecular data. One motivating application is molecular phenotyping using gene expression and other forms of molecular data as predictors of a clinical or physiological state.

The invention addresses the specific context of a binary response Z and many predictors xi; in which the data arises via case-control design, i.e., the numbers of 0/1 values in the response data are fixed by design. This allows for the successful relation of large-scale gene expression data (the predictors) to binary outcomes, such as a risk group or disease state. The invention elaborates on a Bayesian analysis of this particular binary context, with several key innovations.

The analysis of this invention addresses and incorporates case-control design issues in the assessment of association between predictors and outcome with nodes of a tree. With categorical or continuous covariates, this is based on an underlying non-parametric model for the conditional distribution of predictor values given outcomes, consistent with the case-control design. This uses sequences of Bayes' factor based tests of association to rank and select predictors that define significant “splits” of nodes, and that provides an approach to forward generation of trees that is generally conservative in generating trees that are effectively self-pruning. An innovative element of the invention is the implementation of a tree-spawning method to generate multiple trees with the aim of finding classes of trees with high marginal likelihoods, and where the prediction is based on model averaging, i.e., weighting predictions of trees by their implied posterior probabilities. The advantage of the Bayesian approach is that rather than identifying a single “best” tree, a score is attached to all possible trees and those trees which are very unlikely are excluded. Posterior and predictive distributions are evaluated at each node and at the leaves of each tree, and feed into both the evaluation and interpretation tree by tree, and the averaging of predictions across trees for future cases to be predicted.

To demonstrate the utility and advantages of this tree classification model, several embodiments are provided. The first embodiment concerns the prediction of levels of fat content (higher than average versus lower than average) of biscuits based on reflectance spectral measures of the raw dough. The second embodiment concern gene expression profiling using DNA microarray data as predictors of a clinical states in breast cancer. The clinical states include estrogen receptor (“ER”) prediction, tumor recurrence, and lymph node metastases. The example of ER status prediction demonstrates not only predictive value but also the utility of the tree modeling framework in aiding exploratory analysis that identify multiple, related aspects of gene expression patterns related to a binary outcome, with some interesting interpretation and insights. The embodiments also illustrate the use of metagene factors—multiple, aggregate measures of complex gene expression patterns—in a predictive modeling context. The third embodiment relates to the prediction of atherosclerotic phenotype determinative genes. This embodiment is claimed by reference to pending U.S. patent application Ser. No. No. 10/291,885 filed on Nov. 12, 2002, titled “Atherosclerotic Phenotype Determinative Genes and Methods for Using the Same.”

In the case of large numbers of candidate predictors, in particular, model sensitivity to changes in selected subsets of predictors are ameliorated though the generation of multiple trees, and relevant, data-weighted averaging over multiple trees in prediction. The development of formal, simulation-based analyses of such models provides ways of dealing with the issues of high collinearity among multiple subsets of predictors, and challenging computational issues.

The invention also describes a comprehensive modeling approach to combining genomic and clinical data for prediction of disease outcomes in individual patients. Statistical analysis, using predictive classification tree models, evaluates the contributions of multiple forms of data, both clinical and genomic; the latter makes use of metagenes, gene expression signatures derived from microarray analyses. In a breast cancer recurrence study, it is demonstrated that multiple metagenes are far more powerful in predicting outcomes than any single metagene. Furthermore, combining metagenes with clinical risk factors proves most accurate at the individual patient level. This framework for combining multiple forms of data provides a platform for development of models for personalized prognosis.

In one embodiment, the integration of clinical and genomic data has been applied to an initial case study of breast cancer recurrence. The models of the invention incorporate, evaluate and weigh multiple gene expression patterns, clinical factors and treatment regimens in combination, and produce very accurate predictions of recurrence for individual patients. Prediction accuracy assessment includes honestly representing and interpreting uncertainties in prediction—a key emphasis in the modeling approach taught by the invention.

The complexity of the oncogenic process, and of gene-environment interactions that define unique aspects of the course of disease for the individual patient, argue against the view that a simple gene expression profile will accurately predict outcomes for individual patients. Recent examples of gene expression profiling to predict disease recurrence do well in defining broad groups of patients but fall far short of predicting outcomes for an individual. Consistent with this view, that successive sub-categorization of patients according to combinations of both clinical and genomic risk factors highlights the predictive value of multiple genomic patterns in smaller patient subgroups. This combination of risk factors customized to the individual patient level provides accurate predictions of recurrence, and identifies gene patterns and candidates that can now be studied to shed light on potential mechanisms and regulatory pathways. Furthermore, customization of the clinico-genomic integrative model at the individualized patient level, allows for the customization of treatment regimens and development of drug regimens with respect to class of drug, dosage, formulation, and administration with respect to the individual patient.

### BRIEF DESCRIPTION OF THE FIGURES

FIG. 1: An example prediction tree for cookie fat outcomes. The root node splits on predictor/factor 92, followed by two subsequent splits on additional predictors 330 and 305. The Π values are point estimates of the predictive probabilities of high fat versus low fat at each of the nodes, with suffixes simply indexing nodes. The labels Z(0=1) indicate the numbers of low fat (0) and high fat (1) samples within each node, and the F# symbols indicate the thresholds that define the predictor based splits within each node.

FIG. 2: Two predictive factors in cookie dough analysis. All samples are represented by index numbers 1 through 78. Training data are denoted by blue (low fat) and red (high fat), and validation data by cyan (low fat) and magenta (high fat). The two full lines (black) demarcate the thresholds on the two predictors in this example tree.

FIG. 3: Scatter plot of cookie data on three factors in example tree. Samples are denoted by blue (low fat) and red (high fat), with training data represented by filled circles and validation data by open circles.

FIG. 4: Three ER related metagenes in 49 primary breast tumors. Samples are denoted by blue (ER negative) and red (ER positive), with training data represented by filled circles and validation data by open circles.

FIG. 5: Three ER related metagenes in 49 primary breast tumors. All samples are represented by index number in 1-78. Training data are denoted by blue (ER negative) and red (ER positive), and validation data by cyan (ER negative) and magenta (ER positive).

FIG. 6: Honest predictions of ER status of breast tumors. Predictive probabilities are indicated, for each tumor, by the index number on the vertical probability scale, together with an approximate 90% uncertainty interval about the estimated probability. All probabilities are referenced to a notional initial probability (incidence rate) of 0.5 for comparison. Training data are denoted by blue (ER negative) and red (ER positive), and validation data by cyan (ER negative) and magenta (ER positive).

FIG. 7: Cross-validation probability predictions of lymph node status. Samples (tumors) are plotted by index number, and the plotted numbers are marked on the vertical scale at the estimated predictive probabilities of high-risk (red) versus low-risk (blue). Approximate 90% uncertainty intervals about these estimated probabilities are indicated by vertical dashed lines.

FIG. 8: Gene expression patterns from the major metagenes that predict lymph node status. Levels of metagenes for samples are plotted by sample index number and by color (color coding as in FIG. 7).

FIG. 9: Gene expression patterns from the major metagenes that predict lymph node status from current and earlier Duke breast cancer study. Levels of metagenes as in FIG. 8, with current study samples now colored cyan (low-risk) and magenta (high-risk). External validation samples from the 2001 Duke breast cancer study appear as red (high-risk) and blue (low-risk).

FIG. 10: Cross-validation probability predictions of 3-year recurrence. Samples (tumors) are plotted by index number, and the plotted numbers are marked on the vertical scale at the estimated predictive probabilities of 3 year recurrence (red) versus 3 year recurrence free survival (blue). Approximate 90% uncertainty intervals about these estimated probabilities are indicated by vertical dashed lines.

FIG. 11: Cross-validation and external validation probability predictions of lymph node status. Samples (tumors) are plotted by index number, and the plotted numbers are marked on the vertical scale at the estimated predictive probabilities of high-risk versus low risk. Color coding is as in FIG. 9: predictions for the cases in the current study are the same in FIG. 7, but now color coded as magenta (high-risk) and cyan (low risk), the cases from the Duke (PNAS 2001) study are correspondingly color coded red (high-risk) and blue (low-risk). Approximate 90% uncertainty intervals about these estimated probabilities are indicated by vertical dashed lines.

FIG. 12. Kaplan Meier survival curve estimates based on high-low-risk categorization of breast cancer patients on two key metagenes
A. Empirical survival estimates based on the clinical determination of lymph node involvement groupings, labeled LNpos (low-risk: 0-3 positive nodes; high-risk, at least 4 positive nodes).
B. Empirical survival estimates based on a partition into two groups via a threshold on the gene expression pattern of Mg440.
C. Empirical survival estimates showing evidence of interaction between clinical (lymph node status) and genomic (Mg440) factors.
D. Refined empirical survival estimates for two subgroups of the “low Mg440” group, defined by a partition on Mg408.
E. Refined empirical survival estimates for two subgroups of the “high Mg440” group, defined by a partition on Mg109.

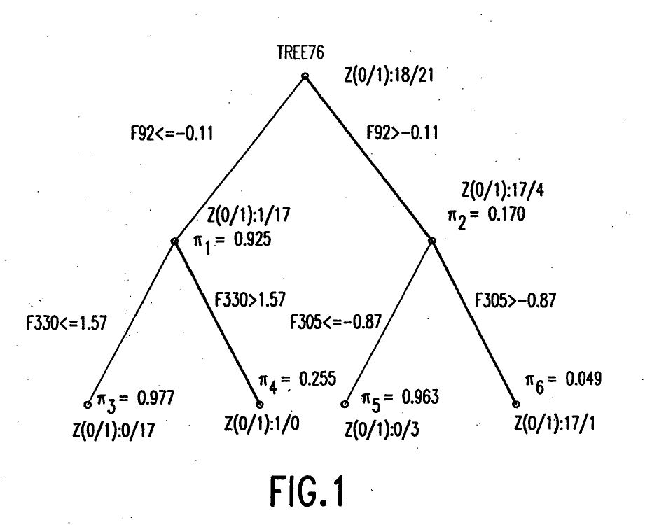

FIG. 13: Use of successive metagene analysis to improve predictions of breast cancer recurrence. Gene expression patterns shown as standard intensity images that relate to splits in the patient sample based on metagene factors. The top image shows the expression pattern of 35 genes of the 117 in Mg440 (the 35 most correlated with Mg440, ordered vertically by correlation with Mg440) on the entire group of 158 patients. Samples are ordered (horizontally) by the value of Mg440, and the vertical black line indicates the threshold on Mg440 defining the optimal split in these trees (threshold of −0.23); this split of patients is that underlying the empirical survival curves in FIG. 1B. The two subgroups of patients defined by this initial split are then further split with two additional metagenes. The group with Mg440 value less than −0.23 (samples 1-61) is further split based on Mg408 and the Mg440 group with value greater than −0.23 (samples 62-158) is split on Mg109. The subsequent two images show the patterns of genes within each of Mg408 and Mg109 for the corresponding two subgroups of patients, arranged similarly within each group and also indicating the second level splits in the tree model. These splits underlie the refined survival curve estimates in FIGS. 12D and 12E. It is evident that, in this traditional format, genes defining these key metagenes clearly show analogue expression patterns that underlie the strong predictive discrimination.

FIG. 14. Predictive genomic and clinico-genomic
A. Metagene tree models. Two of the highest probability trees in analysis of the metagene data alone, showing how metagenes combine to determine successive partitions of the patient sample with associated predictions. The boxes at each node of the tree identify the number of patients and the number under each box is the corresponding model based point estimate of the 4-year recurrence-free probability (given as a percentage) based on the tree model predictions for that group.
B. Clinico-genomic tree models. Two of the highest probability trees illustrating the contribution of lymph node status (lymph node positive count LNpos). Details are as described in panel A.

FIG. 15: Predictor variables in top tree models.
A. Metagene tree models. The figure summarizes the level of the tree in which each variable appears and defines a node split. The numbers on the left simply index trees, and the probabilities in parentheses on the left indicate the relative weights of trees based on fit to the data. The probabilities associated with metagenes (in parentheses on horizontal axis) are sums of the probabilities of trees in which each metagene occurs, and so define overall weights indicating the relative importance of each metagene to the overall model fit and consequent recurrence predictions. Note the appearance of metagenes predictive of ER status (Mg315 and 351) and lymph node metastasis (Mg328 and 408).
B. Clinico-genomic tree models. Predictor variables in top tree models using both clinical data and metagene data. Details are as in Panel A but now the analysis selects from clinical data as well as genomic. Note the appearance of metagenes predictive of lymph node metastasis (Mg408) and Her-2-nu/Erb-b2 status (Mg20). The former is key in the top trees that, defined initially by Mg440, together dominate predictions.

FIG. 16. Honest cross-validation predictions from clinico-genomic tree model.
A. Estimates and approximate 95% confidence intervals for 5-year survival probabilities for each patient. Each patient is honestly predicted in an out-of-sample cross validation based on a model completely regenerated from the data of the remaining patients. Each patient is located on the horizontal axis at the recorded recurrence or censoring time for that patient. Patients indicated in blue are the 5-year recurrence-free cases and those in red are patients that recurred within 5 years. The interval estimates for a few cases that stand out are wide, representing uncertainty due to disparities among predictions coming from individual tree models that are combined in the overall prediction.
B. Estimates and approximate 95% confidence intervals for 4-year survival probabilities for each patient, in the format of panel (A).

FIG. 17. Predicted survival curves for selected patients. Predictive survival curves, and uncertainty estimates for four patients whose clinical and genomic parameters match four actual cases in the data set (cases indexed 15, 158, 98 and 148). Depending on sample sizes within subgroups defined by the tree model analysis, sampling variability, and patterns of “conflict” between the specific set of predictor parameters, the predicted survival curve estimates may have quite substantial associated uncertainties, as indicated by some of these cases. Others, as illustrated, are very much more surely predicted.

### DETAILED DESCRIPTION OF THE INVENTION

I. Development of the Tree Clarification Model: Model Context and Methodology

Data {Zi, xᵢ} (i=1, . . . , n) are available on a binary response variable Z and a p-dimensional covariate vector x: The 0/1 response totals are fixed by design. Each predictor variable xⱼ could be binary, discrete or continuous.

1. Bayes' Factor Measures of Association

At the heart of a classification tree is the assessment of association between each predictor and the response in subsamples, and we first consider this at a general level in the full sample. For any chosen single predictor x; a specified threshold_on the levels of x organizes the data into the 2×2 table.

| | Z = 0 | Z = 1 | |
|---|---|---|---|
| x ≦ τ | n₀₀ | n₀₁ | N₀ |
| x > τ | n₁₀ | n₁₁ | N₁ |
| | M₀ | M₁ | |

With column totals fixed by design, the categorized data is properly viewed as two Bernoulli sequences within the two columns, hence sampling densitie

$$p ( {n}_{0 z} , {n}_{1 z} \vert {M}_{z} , {θ}_{z , τ} ) = {{θ}_{z , τ}^{{n}_{0 z}} ( 1 - {θ}_{z , τ} )}^{{n}_{1 z}}$$

for each column z=0.1. Here, of course, θ_{0,τ}=Pr(x≦τ|Z=0) and θ_{1,τ}=Pr(x≦τ|Z=1). A test of association of the thresholded predictor with the response will now be based on assessing the difference between these Bernoulli probabilities.

The natural Bayesian approach is via the Bayes' factor B_{τ} comparing the null hypothesis θ_{0,τ}θ_{1,τ} to the full alternative θ_{0,τ}≠θ_{1τ}. We adopt the standard conjugate beta prior model and require that the null hypothesis be nested within the alternative. Thus, assuring θ_{0,τ}≠θ_{1,τ}, we take θ_{0,τ}and θ_{1,τ}to be independent with common prior Be(a_{τ}, b_{τ}) with mean m_{τ}=a_{τ}/(a_{τ}+b_{τ}). On the null hypothesis θ_{0,τ}=θ_{1,τ}, the common value has the same beta prior. The resulting Bayes' factor in favour of the alternative over the null hypothesis is then simply

$${B}_{τ} = \frac{β ( {n}_{00} + {a}_{τ} , {n}_{10} + {b}_{τ } ) β ( {n}_{01} + {a}_{τ} \cdot {n}_{11} + {b}_{τ} )}{β ( {N}_{0} + {a}_{τ} , {N}_{1} + {b}_{τ} ) β ( {a}_{τ} , {b}_{τ} )} .$$

As a Bayes' factor, this is calibrated to a likelihood ratio scale. In contrast to more traditional significance tests and also likelihood ratio approaches, the Bayes' factor will tend to provide more conservative assessments of significance, consistent with the general conservative properties of proper Bayesian tests of null hypotheses (See Sellke, T., Bayarri, M. J. and Berger, J. O., Calibration of p_values for testing precise null hypotheses, The American Statistician, 55, 62-71, (2001) and references therein).

In the context of comparing predictors, the Bayes' factor Bτ may be evaluated for all predictors and, for each predictor, for any specified range of thresholds. As the threshold varies for a given predictor taking a range of (discrete or continuous) values, the Bayes' factor maps out a function of τ and high values identify ranges of interest for thresholding that predictor. For a binary predictor, of course, the only relevant threshold to consider is τ=0.

2. Model Consistency with Respect to Varying Thresholds

A key question arises as to the consistency of this analysis as we vary the thresholds. By construction, each probability θ_{Zτ} is a non-decreasing function of τ, a constraint that must be formally represented in the model. The key point is that the beta prior specification must formally reflect this. To see how this is achieved, note first that θ_{Zτ} is in fact the cumulative distribution function of the predictor values χ; conditional on Z=z; (z=0; 1); evaluated at the point χ=τ. Hence the sequence of beta priors, Be(a_{τ}, b_{τ}) as τ varies, represents a set of marginal prior distributions for the corresponding set of values of the cdfs. It is immediate that the natural embedding is in a non-parametric Dirichlet process model for the complete cdf. Thus the threshold-specific beta priors are consistent, and the resulting sets of Bayes' factors comparable as τ varies, under a Dirichlet process prior with the betas as margins. The required constraint is that the prior mean values m_{τ} are themselves values of a cumulative distribution function on the range of χ, one that defines the prior mean of each θ_{τ} as a function. Thus, we simply rewrite the beta parameters (α_{τ}, b_{τ}) as α_{τ}=αₘ_{τ} and b_{τ}=α(1−m_{τ}) for a specified prior mean cdf m_{τ}, and where α is the prior precision (or “total mass”) of the underlying Dirichlet process model. Note that this specialises to a Dirichlet distribution when χ is discrete on a finite set of values, including special cases of ordered categories (such as arise if χ is truncated to a predefined set of bins), and also the extreme case of binary χ when the Dirichlet is a simple beta distribution.

3. Generating a Tree

The above development leads to a formal Bayes' factor measure of association that may be used in the generation of trees in a forward-selection process as implemented in traditional classification tree approaches. Consider a single tree and the data in a node that is a candidate for a binary split. Given the data in this node, construct a binary split based on a chosen (predictor, threshold) pair (χ, τ) by (a) finding the (predictor, threshold) combination that maximizes the Bayes' factor for a split, and (b) splitting if the resulting Bayes' factor is sufficiently large. By reference to a posterior probability scale with respect to a notional 50:50 3 prior, Bayes' factors of 2.2,2.9,3.7 and 5.3 correspond, approximately, to probabilities of 0.9, 0.95, 0.99 and 0.995, respectively. This guides the choice of threshold, which may be specified as a single value for each level of the tree. We have utilised Bayes' factor thresholds of around 3 in a range of analyses, as exemplified below. Higher thresholds limit the growth of trees by ensuring a more stringent test for splits.

The Bayes' factor measure will always generate less extreme values than corresponding generalized likelihood ratio tests (for example), and this can be especially marked when the sample sizes M₀ and M₁ are low. Thus the propensity to split nodes is always generally lower than with traditional testing methods, especially with lower samples sizes, and hence the approach tends to be more conservative in extending existing trees. Post-generation pruning is therefore generally much less of an issue, and can in fact generally be ignored.

The method then incorporates the following steps: Indexing the root node of any tree by zero, and consider the full data set of n observations, representing M_{z} outcomes with Z=z in 0, 1. Labeing1 successive nodes sequentially: splitting the root node, the left branch terminates at node 1, the right branch at node 2; splitting node 1, the consequent left branch terminates at node 3, the right branch at node 4; splitting node 2, the consequent left branch terminates at node 5, and the right branch at node 6, and so forth. Any node in the tree is labelled numerically according to its “parent” node; that is, a node j splits into two children, namely the (left, right) children (2j+1; 2j+2): At level m of the tree (m=0; 1; : : : ;) the candidates nodes are, from left to right, as 2^{m}_{—}1; 2^{m}; : : : ; 2^{m+1}−2.

Having generated a “current” tree, each of the existing terminal nodes are run through one at a time, and assessed as to whether or not to create a further split at that node, stopping based on the above Bayes' factor criterion. Unless samples are very large (thousands) typical trees will rarely extend to more than three or four levels.

4. Inference and Prediction with a Single Tree

Assuming the method generates a tree with m levels, the tree has some number of terminal nodes up to the maximum possible of L=2^{m+1}−2. Inference and prediction involves computations for branch probabilities and the predictive probabilities for new cases that these underlie. This is detailed1 for a specific path down the tree, i.e., a sequence of nodes from the root node to a specified terminal node.

First, the method considers a node j that is split based on a (predictor, threshold) pair labeled (χⱼ, τⱼ), (note that we use the node index to label the chosen predictor, for clarity). It then extends the notation of Section 2.1 to include the subscript j indexing this node. Then the data at this node involves M₀ⱼ cases with Z=0 and M₁ⱼ cases with Z=1. Based on the chosen (predictor, threshold) pair (χⱼ, τⱼ) these samples split into cases n₀₀ⱼ, n₀₁ⱼ, n₁₀ⱼ, n₁₁ⱼ as in the table of Section 2.1, but now indexed by the node label j. The implied conditional probabilities θ_{z,τ,j}=Pr(χⱼ≦τⱼ|Z=z), for z=0, 1 are the branch probabilities defined by such a split (note that these are also conditional on the tree and data subsample in this node, though the notation does not explicitly reflect this for clarity). These are uncertain parameters and, following the development of Section 2.1, have specified beta priors, now also indexed by parent node j, i.e., Be(a_{τ,j}, b_{τ,j}). Assuming the node is split, the two sample Bernoulli setup implies conditional posterior distributions for these branch probability parameters: they are independent with posterior beta distributions
θ_{0,τ,j}˜Be(a_{τ,j}+n₀₀ⱼ, b_{τ,j}+n₁₀ⱼ) and θ_{1,τj}˜Be(a_{τ,j}+n₀₁ⱼ, b_{τ,j}+n₁₁ⱼ).

These distributions allow inference on branch probabilities, and feed into the predictive inference computations as follows.

Consider predicting the response Z* of a new case based on the observed set of predictor values x*. The specified tree defines a unique path from the root to the terminal node for this new case. To predict requires that we compute the posterior predictive probability for Z*=1/0. We do this by following x* down the tree to the implied terminal node, and sequentially building up the relevant likelihood ratio defined by successive (predictor, threshold) pairs.

For example and specificity, suppose that the predictor profile of this new case is such that the implied path traverses nodes 0, 1, 4, 9, terminating at node 9. This path is based on a (predictor, threshold) pair (χ₀, τ₀) that defines the split of the root node, (χ₁, τ₁) that defines the split of node 1, and (χ₄, τ₄) that defines the split of node 4. The new case follows this path as a result of its predictor values, in sequence: (x₀≦τ₀), (x₁>τ₁) and (x₄≦τ₄). The implied likelihood ratio for Z=1 relative to Z=0 is then the product of the ratio of branch probabilities to this terminal node, namely

$${λ}^{*} = \frac{{θ}_{1 , {τ}_{0} , 0}}{{θ}_{0 , {τ}_{0} , 0}} \times \frac{( 1 - {θ}_{1 , {τ}_{1} , 1} )}{( 1 - {θ}_{0 , {τ}_{1} , 1} )} \times \frac{{θ}_{1 , {τ}_{9} , 0}}{{θ}_{0 , {τ}_{9} , 0}} .$$

Hence, for any specified prior probability Pr(Z*=1), this single tree model implies that, as a function of the branch probabilities, the updated probability π* is, on the odds scale, given by

$$\frac{{π}^{*}}{( 1 - {π}^{*} )} = {λ}^{*} \frac{Pr ( {Z}^{*} = 1 )}{Pr ( {Z}^{*} = 0 )} .$$

Hence, for any specified prior probability πPr(Z*=1), this single tree model implies that, as a function the branch probabilities, the updated probability π* is, on the odds scale, given by

$$\frac{{π}^{*}}{( 1 - {π}^{*} )} = {λ}^{*} \frac{Pr ( {Z}^{*} = 1 )}{Pr ( {Z}^{*} = 0 )}$$

The case-control design provides no information about Pr(Z*=1) so it is up to the user to specify this or examine a range of values; one useful summary is obtained by simply taking a 50:50 prior odds as benchmark, whereupon the posterior probability is
πc*=λ*/(1+λ*).

Prediction follows by estimating π* based on the sequence of conditionally independent posterior distributions for the branch probabilities that define it. For example, simply “plugging-in” the conditional posterior means of each θ. will lead to a plug-in estimate of λ* and hence π*. The full posterior for π* is defined implicitly as it is a function of the θ. Since the branch probabilities follow beta posteriors, it is trivial to draw Monte Carlo samples of the θ. and then simply compute the corresponding values of λ* and hence π* to generate a posterior sample for summarization. This way, we can evaluate simulation-based posterior means and uncertainty intervals for π* that represent predictions of the binary outcome for the new case.

5. Generating and Weighting Multiple Trees

In considering potential (predictor, threshold) candidates at any node, there may be a number with high Bayes' factors, so that multiple possible trees with difference splits at this node are suggested. With continuous predictor variables, small variations in an “interesting” threshold will generally lead to small changes in the Bayes' factor—moving the threshold so that a single observation moves from one side of the threshold to the other, for example. This relates naturally to the need to consider thresholds as parameters to be inferred; for a given predictor χ, multiple candidate splits with various different threshold values τ reflects the inherent uncertainty about τ, and indicates the need to generate multiple trees to adequately represent that uncertainty. Hence, in such a situation, the tree generation can spawn multiple copies of the “current” tree, and then each will split the current node based on a different threshold for this predictor. Similarly, multiple trees may be spawned this way with the modification that they may involve different predictors. In problems with many predictors, this naturally leads to the generation of many trees, often with small changes from one to the next, and the consequent need for careful development of tree-managing software to represent the multiple trees. In addition, there is then a need to develop inference and prediction in the context of multiple trees generated this way. The use of “forests of trees” has recently been urged by Breiman, L., Statistical Modeling: The two cultures (with discussion), Statistical Science, 16 199-225 (2001), and our perspective endorses this. The rationale here is quite simple: node splits are based on specific choices of what we regard as parameters of the overall predictive tree model, the (predictor, threshold) pairs. Inference based on any single tree chooses specific values for these parameters, whereas statistical learning about relevant trees requires that we explore aspects of the posterior distribution for the parameters (together with the resulting branch probabilities).

Within the current framework, the forward generation process allows easily for the computation of the resulting relative likelihood values for trees, and hence to relevant weighting of trees in prediction. For a given tree, identify the subset of nodes that are split to create branches. The overall marginal likelihood function for the tree is then the product of component marginal likelihoods, one component from each of these split nodes. Continue with the notation of Section 2.1 but now, again, indexed by any chosen node j: Conditional on splitting the node at the defined (predictor, threshold) pair (χⱼ, τⱼ), the marginal likelihood component is

$${m}_{j} = {\int }_{0}^{1} {\int }_{0}^{1} {\prod }_{z = 0 , 1} p ( {n}_{0 zj} , {n}_{1 zj} \vert {M}_{zj} , {θ}_{z , {τ}_{j} , j} ) p ( {θ}_{z , {τ}_{j} , j} ) ⅆ {θ}_{z , {τ}_{j} , j}$$

while p(θ_{z,τj},j) is the Be(a_{τ,j}, b_{τ,j}) prior for each z=0.1. This clearly reduces to

$${m}_{j} = {\prod }_{z = 0 , 1} \frac{β ( {n}_{0 zj} + {a}_{τ , j} \cdot {n}_{1 zj} + {b}_{τ , j} )}{β ( {a}_{τ , j} , {b}_{τ , j} )} .$$

The overall marginal likelihood value is the product of these terms over all nodes j that define branches in the tree. This provides the relative likelihood values for all trees within the set of trees generated. As a first reference analysis, we may simply normalise these values to provide relative posterior probabilities over trees based on an assumed uniform prior. This provides a reference weighting that can be used to both assess trees and as posterior probabilities with which to weight and average predictions for future cases.

II. Specialized Tree Models Incorporating Multiple Forms of Data: Statistical Tree Models for Survival Time Data with Respect to Breast Cancer Recurrence

The statistical models of the invention can be used for survival time data. In order to aim to evaluate and summarise the regression relationship between multiple, possibly many predictors and the survival time outcomes. In one embodiment, the statistical model can be used for survival time data for relapses/recurrence in breast cancer. The development of the invention uses standard tree model ideas, utilising a Bayesian approach to tree generation, construction, analysis and resulting inference and prediction, and applies the analysis to survival time data.

Survival Distributions for Outcomes

Survival times, such as breast cancer recurrence outcomes following primary surgery, are modelled as arising from conditional survival distributions of Weibull form. This is a flexible class of survival distributions, and in a tree model context it is assumed that each terminal node (or leaf) of any specific tree model is characterized by a specific Weibull distribution particular to that node. If a survival time is denoted t, then we represent t=y^{a} for some Weibull shape parameter and where y is an exponential random variable. The value of is assessed by examining marginal likelihood functions and results discussed are all conditional on a value selected to approximately maximise the marginal likelihood. Hence the model is applied in terms of exponential distributions on the transformed y scale, assuming a specified value of that will be determined in this empirical Bayes' manner.

This results in data {yᵢ, Xᵢ}ᵢ₌₁ where yᵢ is the transformed survival time of individual i and Xᵢ is a p-dimensional vector of covariates. Each predictor variable (each element of Xᵢ) could be categorical or continuous, and the survival times may be right-censored or observed; yᵢ represents the censored time in the latter case, under the assumption of non-informative censoring. Censoring in the breast cancer study is generally due to short-term but continuing follow-up.

Tree Models

A single tree model can be viewed as a recursive partition of a population into refined subgroups based on conjunctions of values of predictor variables. The model is constructed by defining such partitions of the sample data set, and here trees are based on splits of sets of patients according to whether a chosen predictor variable lies above or below a threshold. All predictor variables are considered as candidates for node splits at each node of a tree, and a range of pre-specified threshold values is considered for each predictor. The pre-specified values are taken to span the range of predictor variables at a fairly coarse level. In the examples in breast cancer, metagene data are normalised to zero mean and unit standard deviation, and the grid of thresholds is the quintiles of the empirical distribution across all metagenes, plus the median rounded to zero; categorical clinical predictors are considered for thresholding to categories defined by traditional clinical categories.

At any given node it is possible that any of several (predictor,threshold) pairs would yield a split—as described below—so the ability to generate multiple trees at a node is key. With a continuous predictor a small change in threshold can lead to a change in the resulting model which reflects the uncertainty in the choice of the threshold. The generation of multiple trees is then key in reflecting this uncertainty. So, copies of the “current” tree are made and the current node is split on the predictor but at a different threshold value for each copy. Multiple trees are generated similarly when the (predictor,threshold) pairs involve different predictors as well as different thresholds.

The reported analyses utilise a formal forward-search specification of trees. At a given node of a tree, all possible (predictor,threshold) pairs are considered and evaluated. Pairs that define significant splits are then ranked and the top several chosen; how many splits we consider is limited only by computation. In reported analyses here, we allow up to 10 root node splits and then up to 5 splits of all subsidiary nodes, and generate trees up to a maximum of 5 levels (the root node labeled level 1). Additional constraints to numbers of samples within each node can be considered, though the evaluation using a Bayes' factor test generates a conservative strategy that limits both the proliferation of trees and the depth of any tree, essentially automatically “pruning” the tree.

Bayes' Factor Testing

At any “current” node of a tree, (predictor, threshold) combinations are assessed to split the data at the node into two, more homogeneous subsets based on a standard Bayesian test. With data y₁, . . . , yₙ in this node, and any given single predictor x with a specified threshold τ, the test assesses whether the data are more consistent with a single exponential distribution (with exponential parameter μ) than with two separate exponentials (parameters μ₀ and μ₁) defined by partitioning via x at threshold τ. The Bayesian setup assigns a gamma prior to each of μ, μ₀, μ₁. The prior is Gamma(a, a/m) with mean m. We specify m globally, and treat a as to be estimated, doing so by empirical Bayes' (EB) and then simply utilising the EB estimate of a in the evaluation of the test.

The data summaries can be organised as

| | r | s | |
|---|---|---|---|
| x ≦ τ | r₀ | s₀ | n₀ |
| x ≧ τ | r₁ | s₁ | n₁ |

where r is the number of observed survival times, s the sum of all times (observed and censored), and the (rᵢ, sᵢ) represent the same summaries for the two subsamples. The test of association is based on assessing the Bayes' factor (integrated likelihood ratio) test statistic Bτ (8) to compare the null hypothesis H₀:μ₀=μ₁, taking the common value μ, with the alternative H₁:μ₀≠μ₁. The full model (likelihood and prior) defines H₀ as a null hypothesis properly nested within H₁.

Under the conjugate gamma prior structure,

$$B = \frac{Γ ( α + {r}_{0} ) Γ ( α + {r}_{1} ) }{Γ ( α ) Γ ( α + r ) } \frac{{{α}^{α} ( α + sm )}^{α + r}}{{( α + {s}_{0} m )}^{α + r0} {( α + {s}_{1} m )}^{α + r1}}$$

The Bayes' factor is calibrated to the likelihood-ratio scale. However, it provides more conservative estimates of significance than both likelihood-based approaches and more traditional significance tests such as (See Selke, T., Bayarri, M., and Berger, J. (2001), Calibration of p-values for testing precise null hypotheses, The American Statistician, 55, 62-71). The Bayes' factor will naturally choose smaller models over more complex ones if the quality of fit is comparable and hence provide a control on the size of the trees generated. A useful way to interpret the Bayes' factor is to view B/(1+B) as a reference posterior probability for the split based on a 50:50 prior. Thus, for example, reference probabilities of 0.9 and 0.95 correspond approximately to Bayes' factor values of 9 and 19, respectively. In comparing predictors the Bayes' factor can be evaluated for each predictor at a number of thresholds. This yields a range of values of B which indicate (predictor, threshold) values of interest, and allow us to rank them.

In generating multiple splits at each node of multiple trees a strategy of proliferating trees is adopted. The proliferating trees once constructed are properly compared and evaluated via the likelihood function over trees. Adopting a lower threshold on Bayes' factors (we use B=9 in reported analyses here) leads to more trees than for a higher value, but it is the overall fit of any given tree that is of ultimate interest—relative to other trees and based on its full structure and configuration of the resulting data into subgroups. We may find trees that have individual nodes split at a high level of significance, but that, overall, receive lower weight. Similarly, and more importantly in forward-selection procedures for generating trees, we will generally find trees in which one or more nodes are split at lower levels of significance, but for which the resulting full tree is in fact very much more highly weighted than others. Thus it is important to use a relatively low significance level and then, once multiple trees are generated, sort out which ones are in fact, overall, most significant by evaluating and ranking them according to the tree-model likelihood function (see below).

In most cases a split (parent) node will result in two children nodes. However some non-ordinal categorical predictors may have several categories. The decision to split on such a variable is then based on calculating the Bayes' factor values for all pairwise comparisons among variable levels: a split is made on all levels if the Bayes' factor in one of these comparisons is among the highest across all variables, and exceeds the specified Bayes' factor threshold. A split will result in children nodes which will subsequently define further nodes.

Given a current tree the splitting process continues until either the existing model cannot be improved, i.e., the Bayes' factor criterion is not met at any node, or until all of the remaining candidate split points have few observations. The root node of a tree (level 1) is labeled as node 1 and contains n observations. Nodes are labeled sequentially from left to right; for example, the leftmost branch from the root leads to node 2 while the rightmost branch leads to node 2+k₁−1, where k₁ is the number of children of the root node. These children form level 2 of the tree. The branches from node 2 lead to nodes 2+k₁, . . . , 2+k₂−1 where k₂ is the number of children of node 2 (children located at level 3 of the tree), and so on. As the Bayes' factor criterion is relatively conservative, no post-generation tree pruning is necessary.

Inference in One Tree Model

Suppose a tree with m levels has been generated with a total of L terminal nodes or leaves. Look at (nonterminal) node j of the tree and suppose that it is split on the pair (χⱼ, τⱼ) where j is now the node index. We now need to modify the earlier notation to include the node index. So the number of individuals in node j is now nⱼ; of these, rⱼ individuals have observed survival times and the sum of all survival and censored times is sⱼ. These data are divided at the node, by (χⱼ, τⱼ), yielding n₀ⱼ cases with χ_{j,}≦τⱼ (of which r₀ⱼ cases are observed and with sum of all times s₀ⱼ), and n_{1y} cases with χ_{j,}>τⱼ (of which r₁ⱼ cases are observed and with sum of all times s_{1y}).

Once the node is split, the two resulting exponential parameters have conditional posterior probabilities that are conjugate updates of the Gamma prior. Thus, with the common prior at the parent node Gamma(aj, aj/m) (now indexing the shape parameter, estimated by empirical Bayes' within the node, by j too) posterior gamma distributions are generated as follows:
μ₀ⱼ˜Gamma(aⱼ+r₀ⱼ,aⱼ/m+s₀ⱼ) and μ₁ⱼ˜Gamma(aⱼ+r₁ⱼ/m+s₁ⱼ)
These distributions allow inferences, and feed into predictions, both at nodes in the body of the tree and of course at the terminal nodes (leaves) of the tree. There is “data sharing”, via Bayesian analysis induced shrinkage, between branches at a node since we are utilising all data withing the node to help estimate, via empirical Bayes', the weight parameter aⱼ of the common prior. Thus, for example, in a case where r₀ⱼ is small but r₁ⱼ is larger, it may still be possible to split the node.

Prediction in One Tree Model

Consider now a future case to be predicted—an individual with predictor variables x. The tree defines a single, unique path from the root node to a terminal node (leaf). Prediction requires the evaluation of the posterior (to the training data) predictive distribution for the individual, and can be performed at any node of the tree through which the individual passes, including the root and terminal nodes. Thus, not only as a formal predictive distribution at the terminal node generated, but partial information about how predictions are modified based on the succession of significant node splits on the relevant covariates as they are defined “down the tree are also generated.”

The details are given at the terminal node the individual resides in based on sequential passage dow the tree defined by her predictor variables and the (predictor,threshold) pairs defining the tree. At this node, the model implies a conditional exponential survival time distribution and the corresponding posterior gamma distribution, say Gamma(a*, a*/m*), at the node. The implied (posterior) predictive distribution is then Pareto, implied by integrating the exponential mean with respect to the gamma. This is most easily summarised in terms of the implied survival function, at any point t>0, given by
S(t)=Pr(y>t|x)=(1+m*t/a*)^{-a*}, (t>0).

It is trivial to directly compute point estimates of the predicted survival time for this individual, and quantiles of the distribution to feed into display and interpretation of uncertainties in prediction.

Multiple Trees and Tree Likelihoods

The forward selection procedure can generate hundreds and thousands of trees that then need evaluating and weighting for follow-on inferences and prediction. The invention does this by computing relative likelihood values across trees, which can then be normalised (or weighted by prior probabilities and then normalised) to produce relative posterior probabilities across the set of candidates.

For any single tree the overall marginal likelihood can be calculated, up to a constant, by identifying the terminal nodes (leaves) and computing marginal likelihood components within each and then taking the product. At any one terminal node, suppose there are n cases with r having observed times and the rest censored, and that the sum of all times (censored and uncensored) is s. Then, under the Gamma(a, a/m) prior at that node (with the estimated value of a having been inherited from the parent node, and m specified a priori), the marginal likelihood component is just the integral, with respect to this prior, of the product exponential components (density values for cases with observed times, and survival function values for cases that are right-censored). This standard calculation results in

$$\frac{{a}^{a} {m}^{r}}{{( a + sm )}^{a + r}} \frac{Γ ( a + r )}{Γ ( a )}$$

Taking the product of such terms across all terminal nodes leads to the unnormalised overall marginal likelihood value for the tree. This value is relative to the overall marginal likelihood values of all of the trees generated, which can be normalized to provide relative posterior probabilities for the trees based on an assumed uniform (or other) prior. These probabilities are valuable for both tree assessment and as relative weights in calculating average predictions for future observations.

Prediction Using Multiple Trees

Given a set of trees with normalised tree probabilities based on the above discussion, consider predicting the new case. Index the trees by k, so that we have trees k=1, . . . , K, say, where K may be hundreds. The likelihood values convert to posterior tree probabilities p₁, . . . , pₖ We may choose to ignore very low probability trees in the calculation, so simply restricting to pₖ values above a small threshold and then renormalising (this is of interest for primarily computational reasons since saving many, many unlikely trees has overhead).

In tree k, the individual with predictor variable x has conditional predictive distribution defined by the Pareto result in the unique terminal node where the individual resides; now index that distribution by k, so that, for example, the relevant Pareto survival function is Sₖ(t). Considering all trees, the overall prediction is based on model averaging—theoretically correct and also generally understood to deliver more accurate and reliable predictions that will be generated from any one single, selected model (5; 7)—in this case, any single tree—especially in cases where multiple trees have appreciable probabilities. For example, the survival function can be computed as the simple mixture

$$S ( t ) + {\sum }_{k = 1}^{K} {P}_{k} {S}_{k} ( t ) , ( t > 0 ) .$$

Uncertainty assessments about this “estimated” predictive survival function can be evaluated in a number of ways. Perhaps most direct and easily accessible, as well as most appropriate, is to generate point-wise uncertainty intervals, such as, say, 90% posterior credible intervals around S(t) at a few selected time points t. This is easily derived from a full posterior sample for the survival function at each time point; the value Sₖ(t) is simply the expected value of the exponential survival function exp(−μt) with respect to the relevant gamma prior; so a single random draw from the posterior for the survival function is simply exp(−μt) where the value of μ is sampled from this gamma. Thus, a simulation sample is generated by (a) selecting one of the K components at random, according to the weights pₖ; then (b) drawing the implied μ value and hence the value of the implied exponential survival function; and (c) repeating. The resulting sample can be summarised, in terms of quantiles, for example, to represent uncertainties in predictive survival curves of this mixture form.

III. Collections of Genes and Metagenes Identified by the Invention

The modeling methods of the invention and the analytical methods taught by the invention with respect to clinical, genomic, and biomedical inventions, allow the subject invention to be directed to a collection of genes whose expression is correlated with biological states. In on embodiment, this biological state is a disease state. Such disease states include, but are not limited to cardiovascular diseases such atherosclerosis, breast cancer, and prostate cancer. The invention allows for the identification of any disease state caused by the interactions of multiple genetic and/or clinical factors. In one embodiment, such a disease state is one where multiple, interacting biological and environmental processes define physiological states, and individual dimensions provide only partial information.

Thus, the invention is directed to collections of phenotype determinative genes, as well as methods for using the collection or subparts thereof in various applications. Applications in which the collection finds use, include diagnostic, therapeutic and screening applications. Also reviewed are reagents and kits for use in practicing the subject methods. Finally, a review of various methods of identifying genes whose expression correlates with a given phenotype, such as atherosclerosis and breast cancer is provided.

The subject invention provides a collection of phenotype determinative genes. By phenotype determinative genes is meant genes whose expression or lack thereof correlates with a phenotype. Thus, phenotype determinative genes include genes: (a) whose expression is correlated with the phenotype, i.e., are expressed in cells and tissues thereof that have the phenotype, and (b) whose lack of expression is correlated with the phenotype, i.e., are not expressed in cells and tissues thereof that have the phenotype. A cell is a cell with the indicated phenotype if it is obtained from tissue that is determined to display that phenotype through methods known to those skilled in the art.

The invention claims all collections and subsets thereof of phenotype determinative genes as well as metagenes disclosed herewith. The subject collections of phenotype determinative genes may be physical or virtual. Physical collections are those collections that include a population of different nucleic acid molecules, where the phenotype determinative genes are represented in the population, i.e., there are nucleic acid molecules in the population that correspond in sequence to the genomic, or more typically, coding sequence of the phenotype determinative genes in the collection. In many embodiments, the nucleic acid molecules are either substantially identical or identical in sequence to the sense strand of the gene to which they correspond, or are complementary to the sense strand to which they correspond, typically to an extent that allows them to hybridize to their corresponding sense strand under stringent conditions. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

The nucleic acids that make up the subject physical collections may be single-stranded or double-stranded. In addition, the nucleic acids that make up the physical collections may be linear or circular, and the individual nucleic acid molecules may include, in addition to a phenotype determinative gene coding sequence, other sequences, e.g., vector sequences. A variety of different nucleic acids may make up the physical collections, e.g., libraries, such as vector libraries, of the subject invention, where examples of different types of nucleic acids include, but are not limited to, DNA, e.g., cDNA, etc., RNA, e.g., mRNA, cRNA, etc. and the like. The nucleic acids of the physical collections may be present in solution or affixed, i.e., attached to, a solid support, such as a substrate as is found in array embodiments, where further description of such diverse embodiments is provided below.

Also provided are virtual collections of the subject phenotype determinative genes. By virtual collection is meant one or more data files or other computer readable data organizational elements that include the sequence information of the genes of the collection, where the sequence information may be the genomic sequence information but is typically the coding sequence information. The virtual collection may be recorded on any convenient computer or processor readable storage medium. The computer or processor readable storage medium on which the collection data is stored may be any convenient medium, including CD, DAT, floppy disk, RAM, ROM, etc, which medium is capable of being read by a hardware component of the device.

Also provided are databases of expression profiles of the phenotype determinative genes. Such databases will typically comprise expression profiles of various cells/tissues having the phenotypes, such as various stages of a disease negative expression profiles, prognostic profiles, etc., where such profiles are further described below.

The expression profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc. As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. One format for an output means ranks expression profiles possessing varying degrees of similarity to a reference expression profile. Such presentation provides a skilled artisan with a ranking of similarities and identifies the degree of similarity contained in the test expression profile.

Specific phenotype determinative genes of the subject invention are those listed in the Tables as indicated in the specification. Of the list of genes, certain of the genes have functions that logically implicate them as being associated with the phenotype. However, the remaining genes have functions that do not readily associate them with the phenotype.

The subject invention provides collections of phenotype determinative genes as determined by the methods of the invention. Although the following disclosure describes subject collections in terms of the genes listed in the Tables relevant to each embodiment of the invention described herein, the subject collections and subsets thereof as claimed by the invention apply to all relevant genes determined by the subject invention. Thus, the subject collections and subsets thereof, as well as applications directed to the use of the aforementioned subject collections only serve as an example to illustrate the invention.

The subject collections find use in a number of different applications. Applications of interest include, but are not limited to: (a) diagnostic applications, in which the collections of the genes are employed to either predict the presence of, or the probability for occurrence of, the phenotype; (b) pharmacogenomic applications, in which the collections of genes are employed to determine an appropriate therapeutic treatment regimen, which is then implemented; and (c) therapeutic agent screening applications, where the collection of genes is employed to identify phenotype modulatory agents. Each of these different representative applications is now described in greater detail below.

Diagnostic Applications

In diagnostic applications of the subject invention, cells or collections thereof, e.g., tissues, as well as animals (subjects, hosts, etc., e.g., mammals, such as pets, livestock, and humans, etc.) that include the cells/tissues are assayed to determine the presence of and/or probability for development of, the phenotype. As such, diagnostic methods include methods of determining the presence of the phenotype. In certain embodiments, not only the presence but also the severity or stage of a phenotype is determined. In addition, diagnostic methods also include methods of determining the propensity to develop a phenotype, such that a determination is made that the phenotype is not present but is likely to occur.

In practicing the subject diagnostic methods, a nucleic acid sample obtained or derived from a cell, tissue or subject that includes the same that is to be diagnosed is first assayed to generate an expression profile, where the expression profile includes expression data for at least two of the genes listed in each of the tables relevant to the phenotype. The number of different genes whose expression data, i.e., presence or absence of expression, as well as expression level, that are included in the expression profile that is generated may vary, but is typically at least 2, and in many embodiments ranges from 2 to about 100 or more, sometimes from 3 to about 75 or more, including from about 4 to about 70 or more.

As indicated above, the sample that is assayed to generate the expression profile employed in the diagnostic methods is one that is a nucleic acid sample. The nucleic acid sample includes a plurality or population of distinct nucleic acids that includes the expression information of the phenotype determinative genes of interest of the cell or tissue being diagnosed. The nucleic acid may include RNA or DNA nucleic acids, e.g., mRNA, cRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from a cell or tissue harvested from a subject to be diagnosed, e.g., via biopsy of tissue, using standard protocols, where cell types or tissues from which such nucleic acids may be generated include any tissue in which the expression pattern of the to be determined phenotype exists, including, but not limited, to, monocytes, endothelium, and/or smooth muscle.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile, may be both qualitative and quantitative.

Once the expression profile is obtained from the sample being assayed, the expression profile is compared with a reference or control profile to make a diagnosis regarding the phenotype of the cell or tissue from which the sample was obtained/derived. The reference or control profile may be a profile that is obtained from a cell/tissue known to have an phenotype, as well as a particular stage of the phenotype or disease state, and therefore may be a positive reference or control profile. In addition, the reference or control profile may be a profile from cell/tissue for which it is known that the cell/tissue utlimately developed a phenotype, and therefore may be a positive prognostic control or reference profile. In addition, the reference/control profile may be from a normal cell/tissue and therefore be a negative reference/control profile.

In certain embodiments, the obtained expression profile is compared to a single reference/control profile to obtain information regarding the phenotype of the cell/tissue being assayed. In yet other embodiments, the obtained expression profile is compared to two or more different reference/control profiles to obtain more in depth information regarding the phenotype of the assayed cell/tissue. For example, the obtained expression profile may be compared to a positive and negative reference profile to obtain confirmed information regarding whether the cell/tissue has for example, the diseased, or normal phenotype. Furthermore, the obtained expression profile may be compared to a series of positive control/reference profiles each representing a different stage/level of the phenotype (for example, a disease state), so as to obtain more in depth information regarding the particular phenotype of the assayed cell/tissue. The obtained expression profile may be compared to a prognostic control/reference profile, so as to obtain information about the propensity of the cell/tissue to develop the phenotype.

The comparison of the obtained expression profile and the one or more reference/control profiles may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above.

The comparison step results in information regarding how similar or dissimilar the obtained expression profile is to the control/reference profiles, which similarity/dissimilarity information is employed to determine the phenotype of the cell/tissue being assayed. For example, similarity with a positive control indicates that the assayed cell/tissue has the phenotype. Likewise, similarity with a negative control indicates that the assayed cell/tissue does not have the phenotype.

Depending on the type and nature of the reference/control profile(s) to which the obtained expression profile is compared, the above comparison step yields a variety of different types of information regarding the cell/tissue that is assayed. As such, the above comparison step can yield a positive/negative determination of an phenotype of an assayed cell/tissue. In addition, where appropriate reference profiles are employed, the above comparison step can yield information about the particular stage of the phenotype of an assayed cell/tissue. Furthermore, the above comparison step can be used to obtain information regarding the propensity of the cell or tissue to develop a phenotype.

In many embodiments, the above obtained information about the cell/tissue being assayed is employed to diagnose a host, subject or patient with respect to the presence of, state of or propensity to develop, a disease state. For example, where the cell/tissue that is assayed is determined to have the phenotype, the information may be employed to diagnose a subject from which the cell/tissue was obtained as having the phenotype state, for example, a disease.

Pharmaco/Surgicogenomic Applications

Another application in which the subject collections of phenotype determinative genes find use in is pharmacogenomic and/or surgicogenomic applications. In these applications, a subject/host/patient is first diagnosed for the phenotype, e.g., presence or absence of a disease, propensity to develop the disease, etc., using a protocol such as the diagnostic protocols known to those skilled in the art.

The subject is then treated using a pharmacological and/or surgical treatment protocol, where the suitability of the protocol for a particular subject/patient is determined using the results of the diagnosis step. A variety of different pharmacological and surgical treatment protocols are known to those of skill in the art. Such protocols include, but are not limited to: surgical treatment protocols known to those skilled in the art. Pharmacological protocols of interest include treatment with a variety of different types of agents, including but not limited to: thrombolytic agents, growth factors, cytokines, nucleic acids (e.g. gene therapy agents); etc.

Assessment of Therapy (Therametrics)

Another application in which the subject collections of phenotype determinative genes find use is in monitoring or assessing a given treatment protocol. In such methods, a cell/tissue sample of a patient undergoing treatment for a disease condition is monitored using the procedures described above in the diagnostic section, where the obtained expression profile is compared to one or more reference profiles to determine whether a given treatment protocol is having a desired impact on the disease being treated. For example, periodic expression profiles are obtained from a patient during treatment and compared to a series of reference/controls that includes expression profiles of various phenotype (for example, a disease) stages and normal expression profiles. An observed change in the monitored expression profile towards a normal profile indicates that a given treatment protocol is working in a desired manner.

Therapeutic Agent Screening Applications

The present invention also encompasses methods for identification of agents having the ability to modulate a disease phenotype, e.g., enhance or diminish the phenotype, which finds use in identifying therapeutic agents for a disease. Identification of compounds that modulate a phenotype can be accomplished using any of a variety of drug screening techniques. The screening assays of the invention are generally based upon the ability of the agent to modulate an expression profile of phenotype determinative genes.

The term “agent” as used herein describes any molecule, e.g., protein or pharmaceutical, with the capability of modulating a biological activity of a gene product of a differentially expressed gene. Generally a plurality of assay mixtures are run in parallel with different agent concentrations to obtain a differential response to the various concentrations. Typically, one of these concentrations serves as a negative control, i.e., at zero concentration or below the level of detection. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting differentially expressed gene products) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Exemplary candidate agents of particular interest include, but are not limited to, antisense polynucleotides, and antibodies, soluble receptors, and the like. Antibodies and soluble receptors are of particular interest as candidate agents where the target differentially expressed gene product is secreted or accessible at the cell-surface (e.g., receptors and other molecule stably-associated with the outer cell membrane).

Screening assays can be based upon any of a variety of techniques readily available and known to one of ordinary skill in the art. In general, the screening assays involve contacting a cell or tissue known to have the phenotype with a candidate agent, and assessing the effect upon a gene expression profile made up of phenotype determinative genes. The effect can be detected using any convenient protocol, where in many embodiments the diagnostic protocols described above are employed. Generally such assays are conducted in vitro, but many assays can be adapted for in vivo analyses, e.g., in an animal model of the cancer.

Screening for Drug Targets

In another embodiment, the invention contemplates identification of genes and gene products from the subject collections of determinative genes as therapeutic targets. In some respects, this is the converse of the assays described above for identification of agents having activity in modulating (e.g., decreasing or increasing) a phenotype, and is directed towards identifying genes that are phenotype determinative genes as therapeutic targets.

In this embodiment, therapeutic targets are identified by examining the effect(s) of an agent that can be demonstrated or has been demonstrated to modulate a phenotype (e.g., inhibit or suppress a disease phenotype). For example, the agent can be an antisense oligonucleotide that is specific for a selected gene transcript. For example, the antisense oligonucleotide may have a sequence corresponding to a sequence of a gene appearing in any of the tables relevant to the disease prediction as taught by the instant invention.

Assays for identification of therapeutic targets can be conducted in a variety of ways using methods that are well known to one of ordinary skill in the art. For example, a test cell that expresses or overexpresses a candidate gene, e.g., a gene found in Table 1, is contacted with the known agent, the effect upon a disease phenotype and a biological activity of the candidate gene product assessed. The biological activity of the candidate gene product can be assayed be examining, for example, modulation of expression of a gene encoding the candidate gene product (e.g., as detected by, for example, an increase or decrease in transcript levels or polypeptide levels), or modulation of an enzymatic or other activity of the gene product.

Inhibition or suppression of the disease phenotype indicates that the candidate gene product is a suitable target for therapy. Assays described herein and/or known in the art can be readily adapted in for assays for identification of therapeutic targets. Generally such assays are conducted in vitro, but many assays can be adapted for in vivo analyses, e.g., in an appropriate, art-accepted animal model of the disease state.

Reagents and Kits

Also provided are reagents and kits thereof for practicing one or more of the above described methods. The subject reagents and kits thereof may vary greatly. Reagents of interest include reagents specifically designed for use in production of the above described expression profiles of phenotype determinative genes. One type of such reagent is an array probe nucleic acids in which the phenotype determinative genes of interest are represented. A variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In many embodiments, the arrays include probes for at least 2 of the genes listed in the relevant tables. In certain embodiments, the number of genes that are from the relevant tables that are represented on the array is at least 5, at least 10, at least 25, at least 50, at least 75 or more, including all of the genes listed in the appropriate table. Where the subject arrays include probes for such additional genes, in certain embodiments the number % of additional genes that are represented does not exceed about 50%, usually does not exceed about 25%. In many embodiments a great majority of genes in the collection are phenotype determinative genes, where by great majority is meant at least about 75%, usually at least about 80% and sometimes at least about 85, 90, 95% or higher, including embodiments where 100% of the genes in the collection are phenotype determinative genes. In many embodiments, at least one of the genes represented on the array is a gene whose function does not readily implicate it in the production of the disease phenotype.

Another type of reagent that is specifically tailored for generating expression profiles of phenotype determinative genes is a collection of gene specific primers that is designed to selectively amplify such genes. Gene specific primers and methods for using the same are described in U.S. Pat. No. 5,994,076, the disclosure of which is herein incorporated by reference. Of particular interest are collections of gene specific primers that have primers for at least 2 of the genes listed in Table 1, above. In certain embodiments, the number of genes that are from Table 1 that have primers in the collection is at least 5, at least 10, at least 25, at least 50, at least 75 or more, including all of the genes listed in the relevant table. Where the subject gene specific primer collections include primers for such additional genes, in certain embodiments the number % of additional genes that are represented does not exceed about 50%, usually does not exceed about 25%.

The kits of the subject invention may include the above described arrays and/or gene specific primer collections. The kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like. In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Compounds and Methods for Treatment of a Disease Phenotype

Also provided are methods and compositions whereby relevant disease symptoms may be ameliorated. The subject invention provides methods of ameliorating, e.g., treating, disease conditions, by modulating the expression of one or more target genes or the activity of one or more products thereof, where the target genes are one or more of the phenotype determinative genes as determined by the invention.

Certain cardiovascular diseases and cancers are brought about, at least in part, by an excessive level of gene product, or by the presence of a gene product exhibiting an abnormal or excessive activity. As such, the reduction in the level and/or activity of such gene products would bring about the amelioration of cardiovascular disease symptoms. Techniques for the reduction of target gene expression levels or target gene product activity levels are discussed below.

Alternatively, certain other cardiovascular diseases are brought about, at least in part, by the absence or reduction of the level of gene expression, or a reduction in the level of a gene product's activity. As such, an increase in the level of gene expression and/or the activity of such gene products would bring about the amelioration of cardiovascular disease symptoms. Techniques for increasing target gene expression levels or target gene product activity levels are discussed below.

Compounds that Inhibit Expression, Synthesis or Activity of Mutant Target Gene Activity

As discussed above, target genes involved in relevant disease disorders can cause such disorders via an increased level of target gene activity. A number of genes are now known to be up-regulated in cells/tissues under disease conditions. A variety of techniques may be utilized to inhibit the expression, synthesis, or activity of such target genes and/or proteins. For example, compounds such as those identified through assays described which exhibit inhibitory activity, may be used in accordance with the invention to ameliorate cardiovascular disease symptoms. As discussed, above, such molecules may include, but are not limited to small organic molecules, peptides, antibodies, and the like. Inhibitory antibody techniques are described, below.

For example, compounds can be administered that compete with an endogenous ligand for the target gene product, where the target gene product binds to an endogenous ligand. The resulting reduction in the amount of ligand-bound gene target will modulate endothelial cell physiology. Compounds that can be particularly useful for this purpose include, for example, soluble proteins or peptides, such as peptides comprising one or more of the extracellular domains, or portions and/or analogs thereof, of the target gene product, including, for example, soluble fusion proteins such as Ig-tailed fusion proteins. (For a discussion of the production of Ig-tailed fusion proteins, see, for example, U.S. Pat. No. 5,116,964.). Alternatively, compounds, such as ligand analogs or antibodies that bind to the target gene product receptor site, but do not activate the protein, (e.g., receptor-ligand antagonists) can be effective in inhibiting target gene product activity. Furthermore, antisense and ribozyme molecules which inhibit expression of the target gene may also be used in accordance with the invention to inhibit the aberrant target gene activity. Such techniques are described, below. Still further, also as described, below, triple helix molecules may be utilized in inhibiting the aberrant target gene activity.

Inhibitory Antisense, Ribozyme and Triple Helix Approaches

Among the compounds which may exhibit the ability to ameliorate disease symptoms are antisense, ribozyme, and triple helix molecules. Such molecules may be designed to reduce or inhibit mutant target gene activity. Techniques for the production and use of such molecules are well known to those of skill in the art. Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such within the scope of the invention are engineered hammerhead motif ribozyme molecules that specifically and efficiently catalyze endonucleolytic cleavage of RNA sequences encoding target gene proteins. Specific ribozyme cleavage sites within any potential RNA target are initially identified by scanning the molecule of interest for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays. Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex. Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex. It is possible that the antisense, ribozyme, and/or triple helix molecules described herein may reduce or inhibit the transcription (triple helix) and/or translation (antisense, ribozyme) of mRNA produced by both normal and mutant target gene alleles. In order to ensure that substantially normal levels of target gene activity are maintained, nucleic acid molecules that encode and express target gene polypeptides exhibiting normal activity may be introduced into cells via gene therapy methods such as those described, below, that do not contain sequences susceptible to whatever antisense, ribozyme, or triple helix treatments are being utilized. Alternatively, it may be preferable to co-administer normal target gene protein into the cell or tissue in order to maintain the requisite level of cellular or tissue target gene activity.

Anti-sense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Antibodies for Target Gene Products

Antibodies that are both specific for target gene protein and interfere with its activity may be used to inhibit target gene function. Such antibodies may be generated using standard techniques known in the art against the proteins themselves or against peptides corresponding to portions of the proteins. Such antibodies include but are not limited to polyclonal, monoclonal, Fab fragments, single chain antibodies, chimeric antibodies, etc.

In instances where the target gene protein is intracellular and whole antibodies are used, internalizing antibodies may be preferred. However, lipofectin liposomes may be used to deliver the antibody or a fragment of the Fab region which binds to the target gene epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (e.g., see Creighton, 1983, supra; and Sambrook et al., 1989, supra). Alternatively, single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (Marasco, W. et al., 1993, Proc. Natl. Acad. Sci. USA 90:7889-7893).

In some instances, the target gene protein is extracellular, or is a transmembrane protein. Antibodies that are specific for one or more extracellular domains of the gene product, for example, and that interfere with its activity, are particularly useful in treating cardiovascular disease. Such antibodies are especially efficient because they can access the target domains directly from the bloodstream. Any of the administration techniques described, below which are appropriate for peptide administration may be utilized to effectively administer inhibitory target gene antibodies to their site of action.

Methods for Restoring Target Gene Activity

Target genes that cause the relevant disease may be underexpressed within known disease situations. Several genes are now known to be down-regulated under disease conditions. Alternatively, the activity of target gene products may be diminished, leading to the development of cardiovascular disease symptoms. Described in this section are methods whereby the level of target gene activity may be increased to levels wherein cardiovascular disease symptoms are ameliorated. The level of gene activity may be increased, for example, by either increasing the level of target gene product present or by increasing the level of active target gene product which is present.

For example, a target gene protein, at a level sufficient to ameliorate disease symptoms may be administered to a patient exhibiting such symptoms. Any of the techniques discussed, below, may be utilized for such administration. One of skill in the art will readily know how to determine the concentration of effective, non-toxic doses of the normal target gene protein, utilizing techniques known to those of ordinary skill in the art.

Additionally, RNA sequences encoding target gene protein may be directly administered to a patient exhibiting cardiovascular disease symptoms, at a concentration sufficient to produce a level of target gene protein such that cardiovascular disease symptoms are ameliorated. Any of the techniques discussed, below, which achieve intracellular administration of compounds, such as, for example, liposome administration, may be utilized for the administration of such RNA molecules. The RNA molecules may be produced, for example, by recombinant techniques as is known in the art.

Further, patients may be treated by gene replacement therapy. One or more copies of a normal target gene, or a portion of the gene that directs the production of a normal target gene protein with target gene function, may be inserted into cells using vectors which include, but are not limited to adenovirus, adeno-associated virus, and retrovirus vectors, in addition to other particles that introduce DNA into cells, such as liposomes. Additionally, techniques such as those described above may be utilized for the introduction of normal target gene sequences into human cells. Cells, preferably, autologous cells, containing normal target gene expressing gene sequences may then be introduced or reintroduced into the patient at positions which allow for the amelioration of cardiovascular disease symptoms. Such cell replacement techniques may be preferred, for example, when the target gene product is a secreted, extracellular gene product.

Pharmaceutical Preparations and Methods of Administration

The identified compounds that inhibit target gene expression, synthesis and/or activity can be administered to a patient at therapeutically effective doses to treat or ameliorate the relevant disease. A therapeutically effective dose refers to that amount of the compound sufficient to result in amelioration of symptoms of disease.

Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### Description of the Specific Embodiments

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. All publications mentioned herein are incorporated herein by reference for the purpose of describing and disclosing the subject components of the invention that are described in the publications, which components might be used in connection with the presently described invention.

### EXAMPLE 1

### Analysis of Biscuit Dough Data

A first example concerns the application of biscuit dough data (publicly available at Osborne, B. G., Fearn, T., Miller, A. R. and Douglas, S., Applications of near infrared reflectance spectroscopy to compositional analysis of biscuits and biscuit doughs, J. Sci. Food Agric., 35, 99-105 (1984); Brown, P. J., Fearn, T. and Vannucci, M., The choice of variables in multivariate regression: A non-conjugate Bayesian decision theory approach, Biometrika, 86, 635-648 (1999)) in which interest lies in relating aspects of near infrared (“NIR”) spectra of dough to the fat content of the resulting biscuits. The data set provides 78 samples, of which 39 are taken as training data and the remaining 39 as validation cases to be predicted, precisely as in Brown et al (1999). The binary outcome is 0/1 according to whether the measured fat content exceeds a threshold, where the threshold is the mean of the sample of fat values. As predictors, each xi comprises 300 values of the spectrum of dough sample i, augmented by the set of singular factors (principal components) of the 78 sample spectra, so that p=378; with singular factors indexed 301; : : : ; 378.

The analysis was developed repeatedly exploring aspects of model fit and prediction of the validation sample as the number of control parameters were varied. The particular parameters of key interest varied were the Bayes' factor thresholds that define splits, and controls on the number of such splits that may be made at any one node. It was determined that across ranges of these control parameters, that there was a good degree of robustness. The Bayes' factor threshold was fixed at 3 on the log scale, after which and two-level trees were explored allowing at most 10 splits of the root node and then at most 4 splits of each of nodes 1 and 2. This allowed up to 160 trees, with this analysis generating 148 trees.

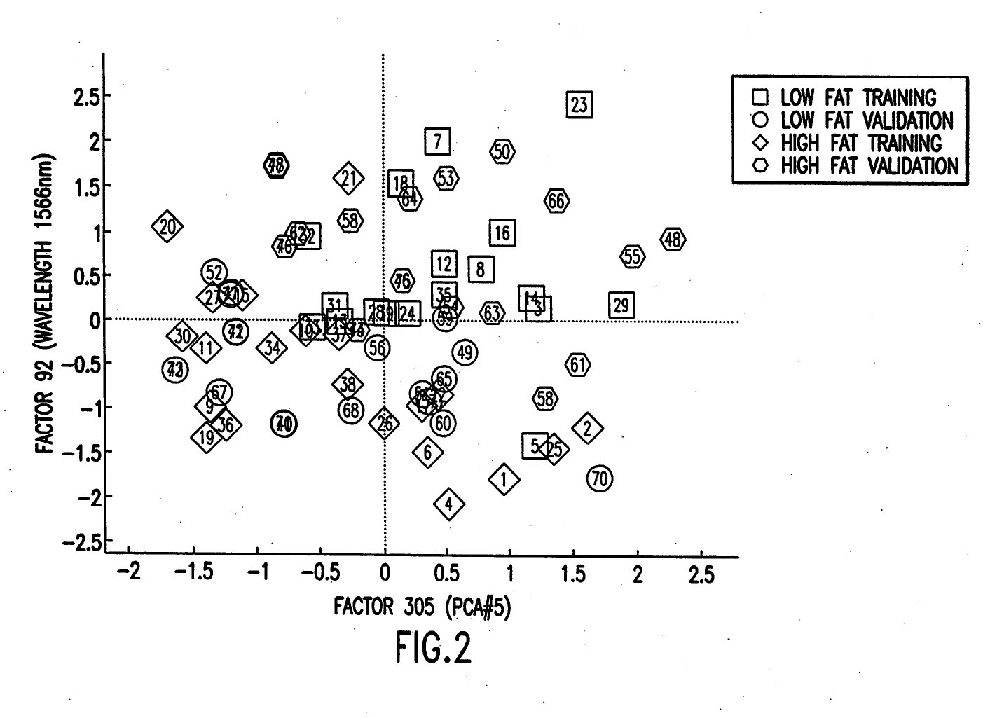

Many of the trees identified had one or two of the predictors in common, and represent variation in the threshold values for those predictors. FIGS. 1-3 display some summaries. FIG. 1 represents one of the 148 trees, split at the root node by the spectral predictor labeled factor 92 (corresponding to a wavelength of 1566 nm). Multiple wavelength values appear in the 148 trees, with values close to this appearing commonly, reflecting the underlying continuity of the spectra. The key second level predictor is factor 305, one of the principal component predictors. The data are scatter plotted on these two predictors in FIG. 2 with corresponding levels of the predictor-specific thresholds from this tree marked.

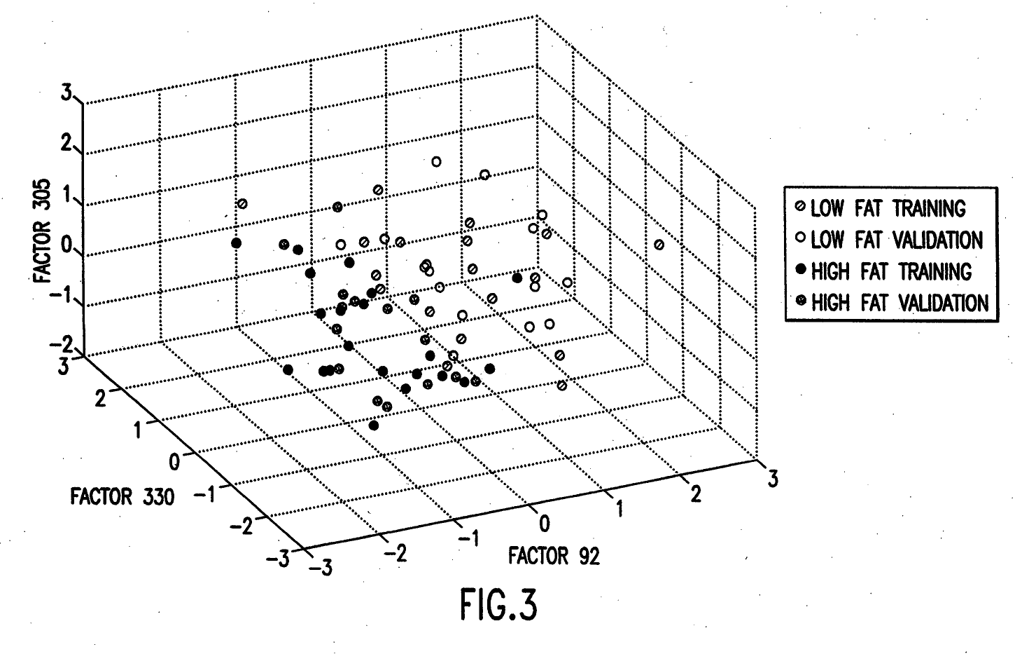

The data appears also against the three predictors in this tree in FIG. 3. Evidently there is substantial overlap in predictor space between the 0/1 outcomes, and cases close to the boundaries defined by any single tree are hard to accurately predict. Nevertheless, in terms of posterior predictive probabilities for the 39 validation samples, accuracy is good. By simply establishing the predictive probability threshold at 0.5 it is determined that 18 of 20 (90%) low fat (blue) cases are “correctly” predicted, as are 19 of 20 (95%) high fat (red) cases.

Predictive accuracy is high in this example with considerable overlap between predictor patterns among the two outcome groups. This is a positive example of the use of the predictive tree approach in a context where standard methods, such as logistic regression, would be less useful. Furthermore, the We end with a note that the 50:50 split of the 78 samples into training and validation sets followed the previous authors as references. Curious about this, we reran the analysis 500 times, each time randomly splitting the data 50:50 into training and validation samples. Predictive accuracy, as measured above, was generally not so good as reported for the initial sample split, varying from a little below 50% to 100% across this set of 500 analyses. The average accuracy for low fat (blue) cases was 80%, and that for high fat (red) cases 76%.

### EXAMPLE 2

### Metagene Expression Profiling to Predict Estrogen Receptor Status of Breast Cancer Tumors

This example illustrates not only predictive utility but also exploratory use of the tree analysis framework in exploring data structure. Here, the tree analysis is used to predict estrogen receptor (“ER”) status of breast tumors using gene expression data. Prior analyses of such data involved binary regression models which utilized Bayesian generalized shrinkage approaches to factor regression. Specifically, prior statistical models involved the use of probit linear regression linking principal components of selected subsets of genes to the binary (ER positive/negative) outcomes. See West, M., Blanchette, C., Dressman, H., Ishida, S., Spang, R., Zuzan, H., Marks, J. R. and Nevins, J. R. Utilization of gene expression profiles to predict the clinical status of human breast cancer. Proc. Natl. Acad. Sci., 98, 11462-11467 (2001). However, the tree model taught in the instant invention presents some distinct advantages over Bayesian linear regression models in the analysis of large non-linear data sets such as these in terms of predictive accuracy and analytical capabilities.

Primary breast tumors from the Duke Breast Cancer SPORE frozen tissue bank were selected for this study on the basis of several criteria. Tumors were either positive for both the estrogen and progesterone receptors or negative for both receptors. Each tumor was diagnosed as invasive ductal carcinoma and was between 1.5 and 5 cm in maximal dimension. In each case, a diagnostic axillary lymph node dissection was performed. Each potential tumor was examined by hematoxylin/eosin staining and only those that were >60% tumor (on a per-cell basis), with few infiltrating lymphocytes or necrotic tissue, were carried on for RNA extraction. The final collection of tumors consisted of 13 estrogen receptor (ER)+lymph node (LN)+tumors, 12 ER LN+tumors, 12 ER+LN tumors, and 12 ER LN tumors.

The RNA was derived from the tumors as follows: Approximately 30 mg of frozen breast tumor tissue was added to a chilled BioPulverizer H tube (Bio101) (Q-Biogene, La Jolla, Calif.). Lysis buffer from the Qiagen (Chatsworth, Calif.) RNeasy Mini kit was added, and the tissue was homogenized for 20 sec in a MiniBeadbeater (Biospec Products, Bartlesville, Okla.). Tubes were spun briefly to pellet the garnet mixture and reduce foam. The lysate was transferred to a new 1.5-ml tube by using a syringe and 21-gauge needle, followed by passage through the needle 10 times to shear genomic DNA. Total RNA was extracted by using the Qiagen RNeasy Mini kit. Two extractions were performed for each tumor, and total RNA was pooled at the end of the RNeasy protocol, followed by a precipitation step to reduce volume. Quality of the RNA was checked by visualization of the 28S:18S ribosomal RNA ratio on a 1% agarose gel. After the RNA preparation, the samples were subject to Affymetrix GENECHIP analysis.

Affymetrix GENECHIP Analysis: The targets for Affymetrix DNA microarray analysis were prepared according to the manufacturer's instructions. All assays used the human HuGeneFL GENECHIP microarray. Arrays were hybridized with the targets at 45° C. for 16 h and then washed and stained by using the GENECHIP Fluidics. DNA chips were scanned with the GENECHIP scanner, and signals obtained by the scanning were processed by GENECHIP Expression Analysis algorithm (version 3.2) (Affymetrix, Santa Clara, Calif.).

A set of n=49 breast cancer samples is analyzed in this study, using predictors based on metagene summaries of the expression levels of many genes. Metagenes, as defined above, are useful aggregate, summary measures of gene expression profiles. The evaluation and summarization of large-scale gene expression data in terms of lower dimensional factors of some form is utilized for two main purposes: first, to reduce dimension from typically several thousand, or tens of thousands of genes to a more practical dimension; second, to identify multiple underlying “patterns” of variation across samples that small subsets of genes share, and that characterize the diversity of patterns evidenced in the full sample. Although, the analysis is conducive to the use of various factor model approaches known to those skilled in the art, a cluster-factor approach is used here to define empirical metagenes. This defines the predictor variables x utilized in the tree model. Metagenes can be obtained by combining clustering with empirical factor methods. The metagene summaries used in the ER example in this disclosure, are based on the following steps.
Assume a sample of n profiles of p genes;
Screen genes to reduce the number by eliminating genes that show limited variation across samples or that are evidently expressed at low levels that are not detectable at the resolution of the gene expression technology used to measure levels. This removes noise and reduces the dimension of the predictor variable;
Cluster the genes using k_means, correlated-based clustering. Any standard statistical package may be used. This analysis uses the xcluster software created by Gavin Sherlock (http://genomewww.stanford.edu/sherlock/cluster.html). A large number of clusters are targeted so as to capture multiple, correlated patterns of variation across samples, and generally small numbers of genes within clusters;
Extract the dominant singular factor (principal component) from each of the resulting clusters. Again, any standard statistical or numerical software package may be used for this; this analysis uses the efficient, reduced singular value decomposition function (“SVD”) in the Matlab software environment (http://www.mathworks.com/products/matlab).

In the analysis of the ER data in this disclosure, the original data was developed using Affymetrix arrays with 7129 sequences, of which 7070 were used (following removal of Affymetrix controls from the data.). The expression estimates used were log2 values of the signal intensity measures computed using the dChip software for post-processing Affymetrix output data (See Li, C. and Wong, W. H. Model-based analysis of oligonucleotide arrays: Expression index computation and outlier detection. Proc. Natl. Acad. Sci., 98, 31-36 (2001), and the software site http://www.biostat.harvard.edu/complab/dchip/). With a target of 500 clusters, the xcluster software implementing the correlation-based k_means clustering produced p=491 clusters. The corresponding p metagenes were then evaluated as the dominant singular factors of each of these clusters, as referenced above. See Table that provide tables detailing the 491 metagenes.

The data comprised 40 training samples and 9 validation cases. Among the latter, 3 were initial training samples that presented conflicting laboratory tests of the ER protein levels, so casting into question their actual ER status; these were therefore placed in the validation sample to be predicted, along with an initial 6 validation cases selected at random. These three cases are numbers 14, 31 and 33. The color coding in the graphs is based on the first laboratory test (immunohistochemistry). Additional samples of interest are cases 7, 8 and 11, cases for which the DNA microarray hybridizations were of poor quality, with the resulting data exhibiting major patterns of differences relative to the rest.

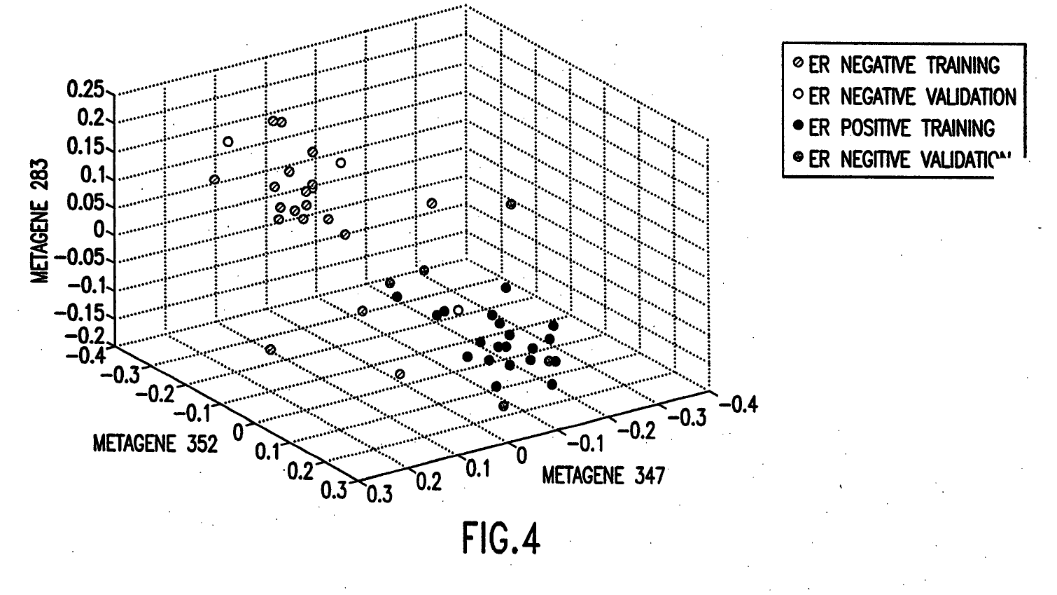

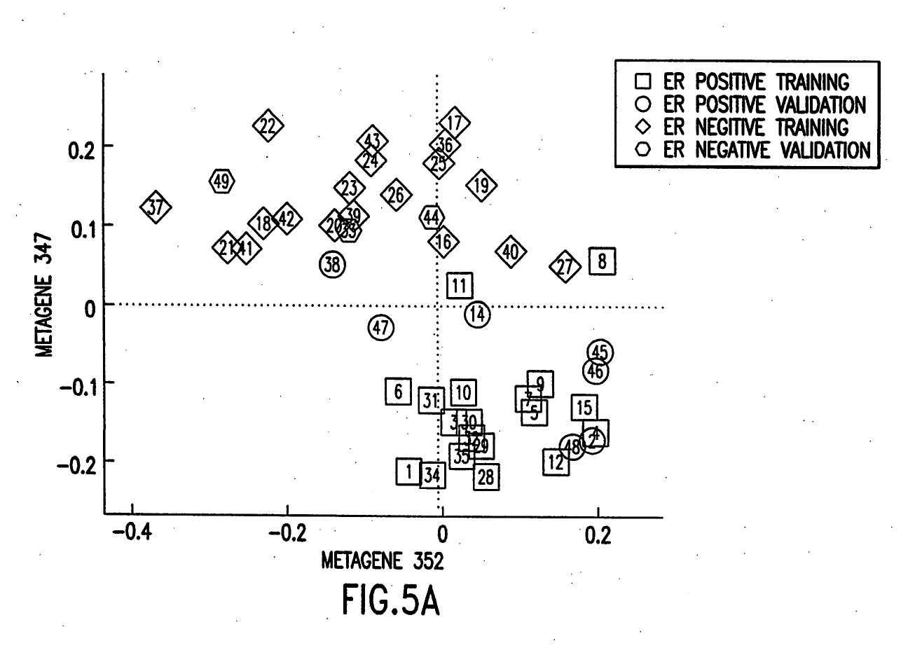

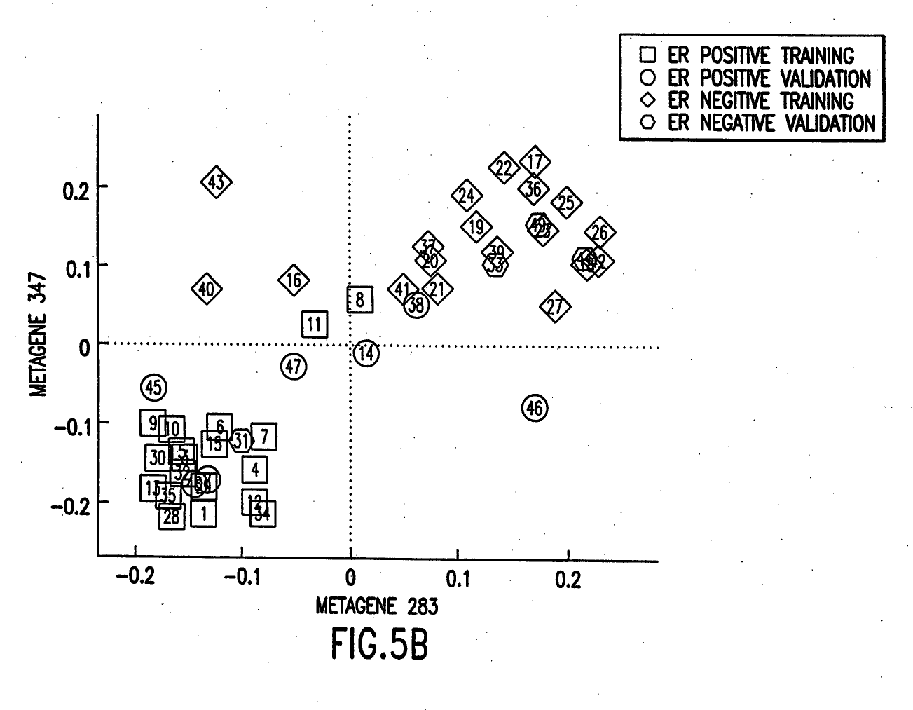

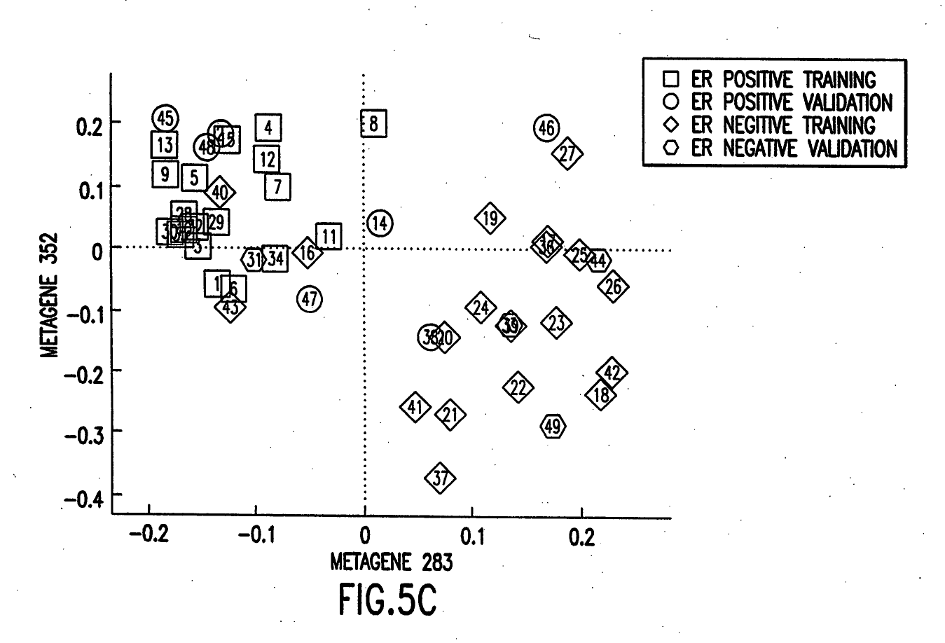

The metagene predictor has dimension p=491: the analysis generated trees based on a Bayes' factor threshold of 3 on the log scale, allowing up to 10 splits of the root node and then up to 4 at each of nodes 1 and 2. Some pertinent summaries appear in the following figures. FIGS. 4 and 5 display 3-D and pairwise 2-D scatter plots of three of the key metagenes, all clearly strongly related to the ER status and also correlated. However, there are in fact five or six metagenes that quite strongly associate with ER status and it is evident that they reflect multiple aspects of this major biological pathway in breast tumors. In the study reported in West et al (2001), Bayesian probit regression models were utilized with singular factor predictors which identified a single major factor predictive of ER. That analysis identified ER negative tumors 16, 40 and 43 as difficult to predict based on the gene expression factor model; the predictive probabilities of ER positive versus negative for these cases were near or above 0.5, with very high uncertainties reflecting real ambiguity.

In contrast to the more more traditional regression models, the current tree model identifies several metagene patterns that together combine to define an ER profile of tumors, and that when displayed as in FIGS. 4 and 5 isolate these three cases as quite clearly consistent with their designated ER negative status in some aspects, yet conflicting and much more in agreement with the ER positive patterns on others. Metagene 347 is the dominant ER signature; the genes involved in defining this metagene include two representations of the ER gene, and several other genes that are coregulated with, or regulated by, the ER gene. Many of these genes appeared in the dominant factor in the regression prediction. This metagene strongly discriminates the ER 11 negatives from positives, with several samples in the mid-range. Thus, it is no surprise that this metagene shows up as defining root node splits in many high-likelihood trees. This metagene also clearly defines these three cases—16, 40 and 43—as appropriately ER negative. However, a second ER associated metagene, number 352, also defines a significant discrimination. In this dimension, however, it is clear that the three cases in question are very evidently much more consistent with ER positives; a number of genes, including the ER regulated PS2 protein and androgen receptors, play roles in this metagene, as they did in the factor regression; it is this second genomic pattern that, when combined together with the first as is implicit in the factor regression model, breeds the conflicting information that fed through to ambivalent predictions with high uncertainty.

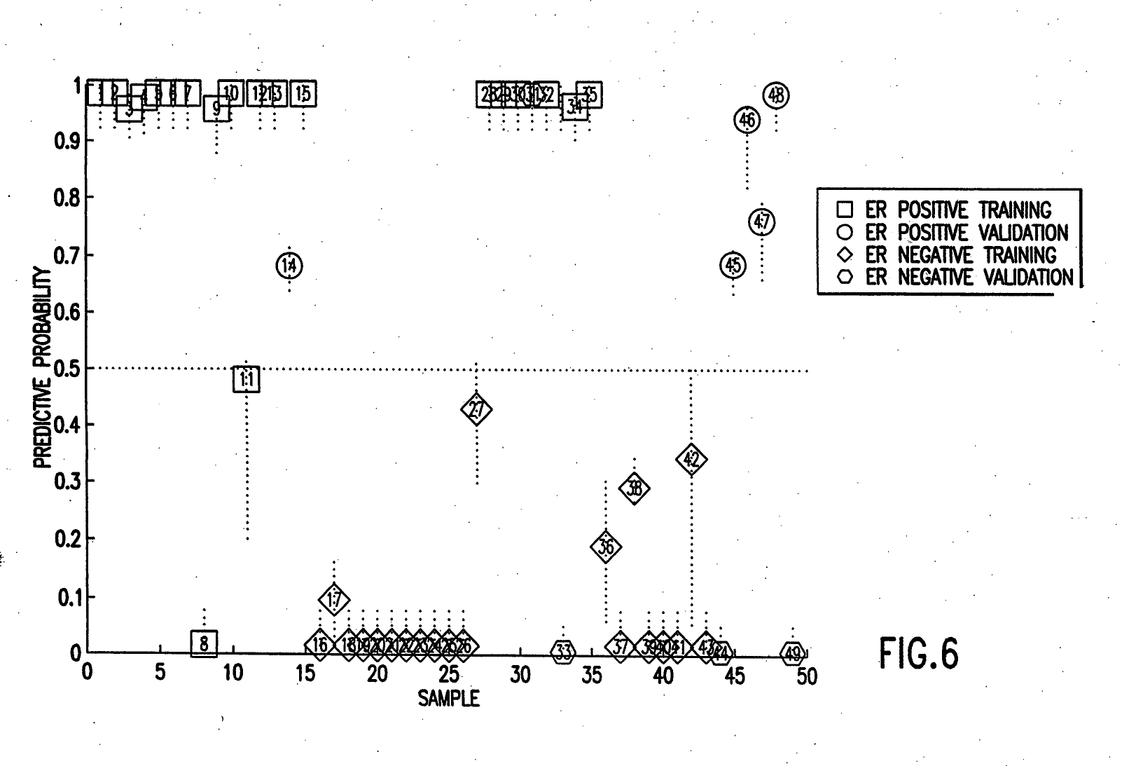

The tree model analysis here identifies multiple interacting patterns and allows easy access to displays such as those shown in FIGS. 4 to 6 that provide insights into the interactions, and hence to interpretation of individual cases. In the full tree analysis, predictions based on averaging multiple trees are in fact dominated by the root level splits on metagene 347, with all trees generated extending to two levels where additional metagenes define subsidiary branches. Due to the dominance of metagene 347, the three interesting cases noted above are perfectly in accord with ER negative status, and so are well predicted, even though they exhibit additional, subsidiary patterns of ER associated behaviour identified in the figures. FIG. 6 displays summary predictions. The 9 validation cases are predicted based on the analysis of the full set of 40 training cases. Predictions are represented in terms of point predictions of ER positive status with accompanying, approximate 90% intervals from the average of multiple tree models. The training cases are each predicted in an honest, cross-validation sense: each tumor is removed from the data set, the tree model is then refitted completely to the remaining 39 training cases only, and the hold-out case is predicted, i.e., treated as a validation sample. Excellent predictive performance is observed for both these one-at-a-time honest predictions of training samples and for the out of sample predictions of the 9 validation cases. One ER negative, sample 31, is firmly predicted as having metagene expression patterns completely consistent with ER positive status. This is in fact one of the three cases for which the two laboratory tests conflicted. The other two such cases, however agree with the initial ER negative test result—number 33, for which the predictions firmly agree with the initial ER negative test result, and number 14, for which the predictions agree with the initial ER positive result though not quite so forcefully. The lack of conformity of expression patterns in some cases (Case 8, 11 and 7) are due to major distortions in the data on the DNA microarray due to hybridization problems.

### EXAMPLE 3A

### Prediction of Lymph Node Metastases and Cancer Recurrence

This study assesses complex, multivariate patterns in gene expression data from primary breast tumor samples that can accurately predict nodal metastatic states and relapse for the individual patient using the statistical tree model of the invention. DNA microarray data on samples of primary breast tumors was generated to which non-linear statistical analyses embodied by the tree model of the invention was applied to evaluate multiple patterns of interactions of groups of genes that have true predictive value, at the individual patient level, with respect to lymph node metastasis and cancer recurrence. For both lymph node metastasis and cancer recurrence, patterns of gene expression (metagenes) were identified that associate with outcome.

Much more importantly, these patterns were capable of honestly predicting outcomes in individual patients with about 90% accuracy, based on a simple threshold of 0.5 probability in each case. The metagenes that predict lymph node metastasis and recurrence identify distinct groups of genes, suggesting different biological processes underlying these two characteristics of breast cancer.

Patients and biopsy specimens: The analyses of gene expression phenotypes drew samples from 171 primary tumor biopsies at the Koo Foundation Sun Yat-Sen Cancer Center (KF-SYSCC) in Taipei, Taiwan, collected and banked from 1991 to 2001. Samples from eleven patients who received preoperative chemotherapy and one with in-situ carcinoma were excluded from analysis. These 159 samples represent a heterogeneous population, though patient selection was enriched with cases of longer-term follow-up and observed recurrences. For a final analysis, only 89 samples were used. The median follow-up was 49 months. Full details of clinical characteristics are shown in Table 1.

Microarray analysis: Tumor total RNA was extracted with Qiagen RNEasy kits, and assessed for quality with an Agilent Lab-on-a-Chip 2100 Bioanalyzer. Hybridization targets were prepared from total RNA according to Affymetrix protocols and hybridized to Affymetrix Human U95 GeneChip arrays See West M, Blanchette C, Dressman H, Huang E, Ishida S, Spang R et al. Predicting the clinical status of human breast cancer by using gene expression profiles, Proc Natl Acad Sci, 98:11462-11467 (2001).

Hybridization procedures and parameters. The amount of starting total RNA for each reaction was 20 μmcg. Briefly, first strand cDNA synthesis was generated using a T7-linked oligo-dT primer, followed by second strand synthesis. An in vitro transcription reaction was performed to generate the cRNA containing biotinylated UTP and CTP, which was subsequently chemically fragmented at 95° C. for 35 min. The fragmented, biotinylated cRNA was hybridized in MES buffer (2-[N-morpholino]ethansulfonic acid) containing 0.5 mg/ml acetylated bovine serum albumin to Affymetrix GeneChip Human U95Av2 arrays at 45° C. for 16 hr, according to the Affymetrix protocol (www.affymetrix.com and www.affymetrix.com/products/arrays/specific/hgu95.affk). The arrays contain over 12,000 genes and ESTs. Arrays were washed and stained with streptavidin-phycoerythrin (SAPE, Molecular Probes). Signal amplification was performed using a biotinylated anti-streptavidin antibody (Vector Laboratories, Burlingame, Calif.) at 3 μmcg/ml. This was followed by a second staining with SAPE. Normal goat IgG (2 mg/ml) was used as a blocking agent.

Measurement data and specifications. Scans were performed with an Affymetrix GeneChip scanner and the expression value for each gene was calculated using the Affymetrix Microarray Analysis Suite (v5.0), computing the expression intensities in ‘signal’ units defined by software. Scaling factors were determined for each hybridization based on an arbitrary target intensity of 500. Scans were rejected if the scaling factor exceeded a factor of 25, resulting in only one reject. Array design. All assays employed the Affymetrix Human U95Av2 GeneChip. The characteristics of the array are detailed on the Affymetrix web site (www.affymetrix.com/products/arrays/specific/hgu95.affx).

Statistical analysis: This analysis used the predictive statistical tree model of this invention. The method of the invention first screens genes to reduce noise, applies k-means correlation-based clustering targeting a large number of clusters, and then uses singular value decompositions (“SVD”) to extract the single dominant factor (principal component) from each cluster. This generated 496 cluster-derived singular factors (metagenes) that characterize multiple patterns of expression of the genes across samples. The strategy aimed to extract multiple such patterns while reducing dimension and smoothing out gene-specific noise through the aggregation within clusters. Formal predictive analysis then uses these metagenes in a Bayesian classification tree analysis. This generates multiple recursive partitions of the sample into subgroups (the “leaves” of the classification tree), and associates Bayesian predictive probabilities of outcomes with each subgroup. Overall predictions for an individual sample are then generated by averaging predictions, with appropriate weights, across many such tree models. Iterative out-of-sample, cross-validation predictions are then performed leaving each tumor out of the data set one at a time, refitting the model from the remaining tumors and using it to predict the hold-out case. This rigorously tests the predictive value of a model and mirrors the real-world prognostic context where prediction of new cases as they arise is the major goal. Although, clinico-pathologic parameters such as the presence or absence of positive axillary nodes represent the best means available to classify patients into broad subgroups by recurrence and survival, such methods remain an imperfect tool. Among patients with no detectable lymph node involvement, a population thought to be in a low risk category, between 22 and 33% develop recurrent disease after a 10-year follow-up. See Polychemotherapy for early breast cancer: an overview of the randomized trials, Early Breast Cancer Trialists' Collaborative Group, Lancet; 352:930-942 (2001). Thus, properly identifying individuals out of this group who are at risk for recurrence is beyond the current capabilities of most predictive diagnostics. Details of the statistical analysis as taught by the instant invention are as follows:
Raw data are the 12,625 signal intensity measures of expression of genes on the Affymetrix HU95aV2 DNA microarray, with signal intensities based on the Affymetrix V5 software then transformed to the log-base 2 scale. An initial screen reduces this to a total of 7,030 genes to remove sequences that vary at low levels or minimally. Specifically, this screens out genes whose expression levels across all samples varies by less than two-fold, and whose maximum signal intensity value is lower than nine on a log-base 2 scale.
The set of samples on these 7,030 genes are clustered using k-means correlated-based clustering. Any standard statistical package may be used for this; our analysis uses the xcluster software created by Gavin Sherlock at Stanford University (http://genome-www.stanford.edu/sherlock/cluster.html). We defined a target of 500 clusters and the xcluster routine delivered 496 in this analysis.
The dominant singular factor (principal component) from each of the 496 clusters is extracted. Again, any standard statistical or numerical software package may be used for this; this analysis uses the reduced singular value decomposition function (svd) in Matlab. (http://www.mathworks.com/products/matlab).
These 496 metagene predictors are input to the tree model analysis. A key ingredient is the generalized likelihood ratio, or Bayes' factor, measure of association between metagenes and binary outcomes. An initial ordering of metagenes is provided by the Bayes' factor values on all the data (at the root node of the tree). “Top” metagenes are those with highest Bayes' factor in this sense, and several “top” metagenes were selected to define the lists of genes (accompanying material) as described further below. Specifics parameters defined to create the precise tree models in the two breast examples are as follows. The tree model analysis as reported utilised a Bayes' factor threshold of 3 on the log scale, allowed up to 10 splits of the root node and then up to 4 at each of nodes 1 and 2. Trees were allowed to grow to at most 2 levels consistent with the relatively small sample size of the data sets.
Predictions for individual patients were performed as described in the paper: the analysis was repeated for each patient, holding out from the model fitting the expression and outcome data for that patient, and then developing the statistical tree model analysis based on only the remaining data. Then, the hold-out patient was predicted. We note that the model fitting, including the statistical evaluation of which metagenes are most predictive and the roles they play in the analysis (i.e., the “feature selection process”) is repeated anew for each of these analyses. Were this not done, and metagene selection based on all the data, then the predictions would appear much more accurate, but incorrectly and misleadingly so. This critical perspective, which we have terms “honest prediction” in the cross-validation context, is one we have taken pains to stress in our work (e.g., reference 11) and one that defines our approach to critical model evaluation when prediction is a primary focus.
The lists of genes were generated precisely as follows, for each of the recurrence and metastasis analyses separately. From the statistical tree model fit to all the data, the “top” 4 metagenes were selected, based on the marginal Bayes' factor association measure as described. This defines 4 clusters of genes that are the initial basis of the list. The list was extended by adding in additional genes that are most highly correlated (standard linear correlation) with each of these 4 metagenes; the set of unique genes in the resulting lists are reported and form part of this supplementary material, as are full details of all genes defining each of the 496 metagenes.
In the lymph node metastasis external validation test, the predictions of the sample of cancers from the Duke 2001 PNAS study were performed directly using the tree model fitted only to the data from the current study (as described). That is, predictions were performed entirely out-of-sample with no modification at all to the definition of metagenes, the model or the details of analysis, so paralleling the “real life” circumstances of predicting new patients and providing a completely honest out-of-sample assessment of generalization and predictive validity.
The metagene data for the Duke breast cancer samples used for external validation via out-of-sample prediction were evaluated as follows. The samples are from a 2000 study and gene expression profiles are on the early Affymetrix HU6800 array. The first step was then to identify all genes on that array (7,129 genes) that are also represented among the 12,625 genes on the U95av2 array. This was done using the chip-to-chip key available at the Affymetrix web site. This allows for the identification of genes on the HU6800 array that map to genes within each of the 496 metagene clusters from the current study. For example, the key metagenes 330, 146 and 130 have precisely 30, 37 and 8 genes, respectively; mapping these genes to the earlier HU6800 array identifies sets of 26, 42 and 4 genes, respectively (note that there are duplicates in some cases, as for metagene 146 here). These sets of genes on the HU6800 array define the metagene clusters and the corresponding value of the metagenes are evaluated precisely as described, using the dominant singular factor (principal component) from each of the 496 clusters.

The question of lymph node diagnosis is part of the broader issue of more accurately predicting breast cancer disease course and recurrence. Recently, genomic-scale measures of gene expression, using microarrays and other technologies have opened a new avenue for cancer diagnosis. They identify patterns of gene activity that sub-classify tumors, and such patterns may correlate with the biological and clinical properties of the tumors. The utility of such data in improving prognosis will relies on analytical methods that accurately predict the behavior of the tumors based on expression patterns. Credible predictive evaluation is critical in establishing valid and reproducible results and implicating expression patterns that do indeed reflect underlying biology. This predictive perspective is a key step towards integrating complex data into the process of prognosis for the individual patient, a step that can be accomplished through the practice of the present invention.

Furthermore, an ultimate goal is to integrate molecular and genomic information with traditional clinical risk factors, including lymph node status, patient age, hormone receptor status, and tumor size, in comprehensive models for predicting disease outcomes. Rather than supplant traditional clinical appraisal, genomic data adds data to traditional risk factors, and assessing individuals based on combinations of relevant traditional risk factors with identified genomic factors could potentially improve predictions. The present invention allows this goal to be realized by demonstrating the ability of genomic data to accurately predict lymph node involvement and disease recurrence in defined patient subgroups. Most importantly, these predictions are relevant for the individual patient and can provide a quantitative measure of the probability for the clinical phenotype and outcome of disease. Such predictions may ultimately facilitate treating patients as individuals rather than as unidentifiable members of a risk profile as described in the following examples.

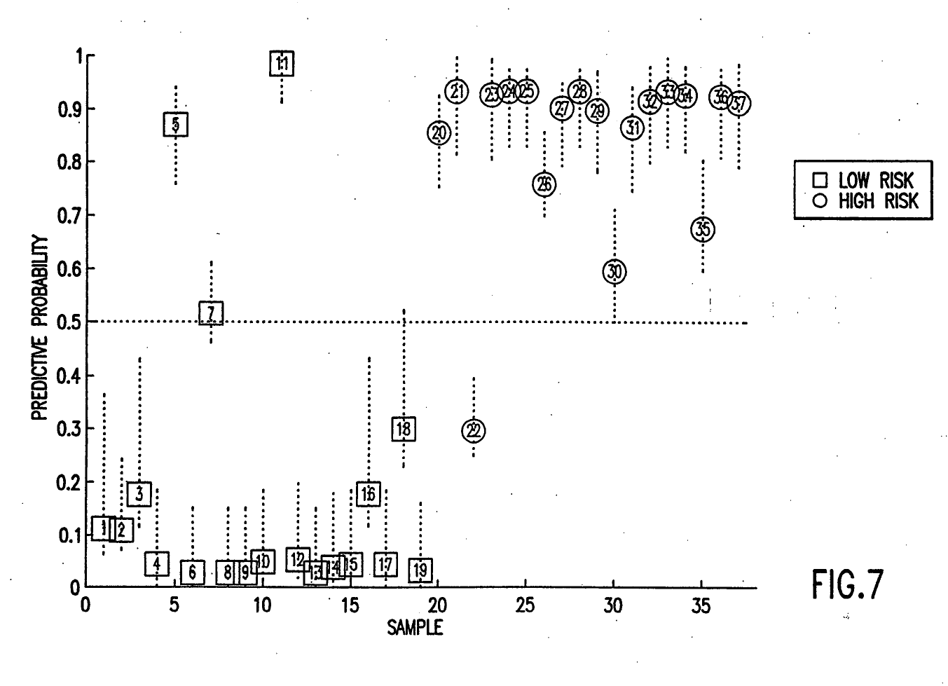

The present invention was applied to the analysis of gene expression patterns in primary breast tumors that predict lymph node metastasis, as well as tumor recurrence. The first study compares traditional “low-risk” versus “high-risk” patients, primarily based on age, primary tumor size, lymph node status, and Estrogen receptor (“ER”) status. Among ER positive individuals, the “high-risk” clinical profile is represented by advanced lymph node metastases (10 or more positive nodes); the “low-risk profile” identifies node-negative women of age greater than 40 years with tumor size below 2 cm. The number of samples in the tumor collection that met these criteria reduced down to 18 high-risk and 19 low-risk cases (37 of the 89 samples in Table 1). Expression data were generated and metagenes identified and used in the Bayesian statistical tree analysis. FIG. 7 displays summary predictions from the resulting total of 37 cross-validation analyses. For each individual tumor, this graph illustrates the predicted probability for “high-risk” versus “low-risk” (red versus blue) together with an approximate 90% confidence interval, based on analysis of the 36 remaining tumors performed successively 37 times as each tumor prediction is made. It is important to recognize that each sample in the data set, when assayed in this manner, constitutes a validation set that accurately assesses the robustness of the predictive model. The metagene model accurately predicts metastatic potential; about 90% of cases are accurately predicted based on a simple threshold at 0.5 on the estimated probability in each case. Case number 7 is in the intermediate zone, exhibiting patterns of expression of the selected metagenes that relate equally well to those of “high-” and “low-risk” cases, while case 22 is a clinical “high-risk” case with genomic expression patterns that relate more closely to “low-risk” cases. In contrast, node negative patients 5 and 11 have gene expression patterns more strongly indicative of “high-risk”, and are key cases for follow-up investigations. The details of clinical information in these apparently discordant cases are shown in Table 2.

Clinical features of these “discordant” cases are illuminating, and suggestive of how a broader investigation of clinical data combined with molecular model-based predictions may aid in the eventual decision-making process. Although case 22 did in fact recur, 6 years post-surgery; this patient's clinical classification as high risk for recurrence based on purely clinical parameters was moderated by a lower risk based on metagenes, as demonstrated by this patient having survived recurrence-free for a longer time. Thus the lower probability prediction assigned to patient 22 based on the gene expression profiles is reflected in the clinical behavior of her disease. The “low-risk” patient 7 recurred at 31 months, and patient 11 at 38 months, whereas case 5 is currently disease-free after only 12 months of follow-up. Again, case 7, and to some degree case 11, thus partly corroborate the predictions based on genomic criteria. data. With such predictions as part of a prognostic model, more intensive or innovative post-surgical therapy should perhaps have been recommended for these two cases.

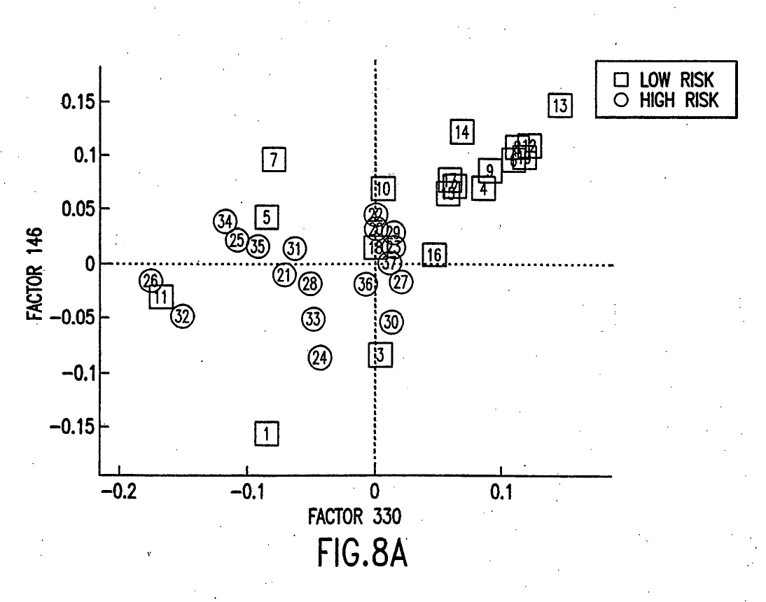

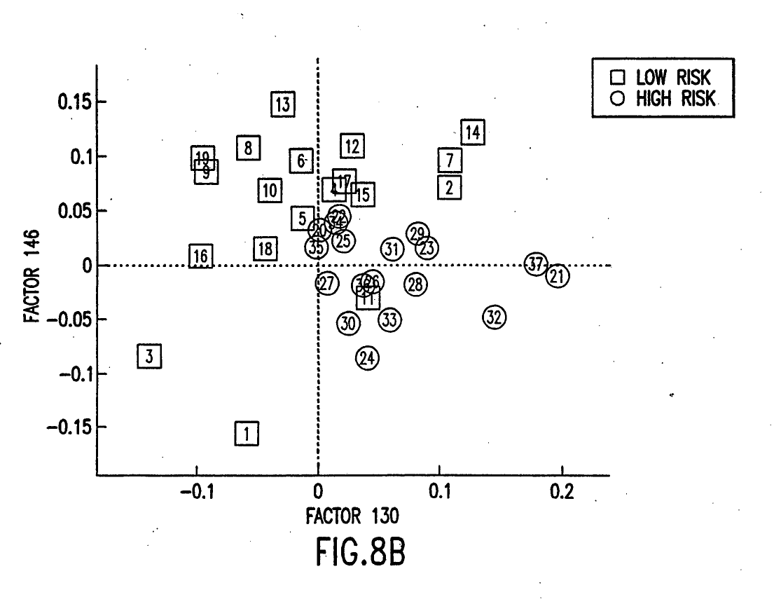

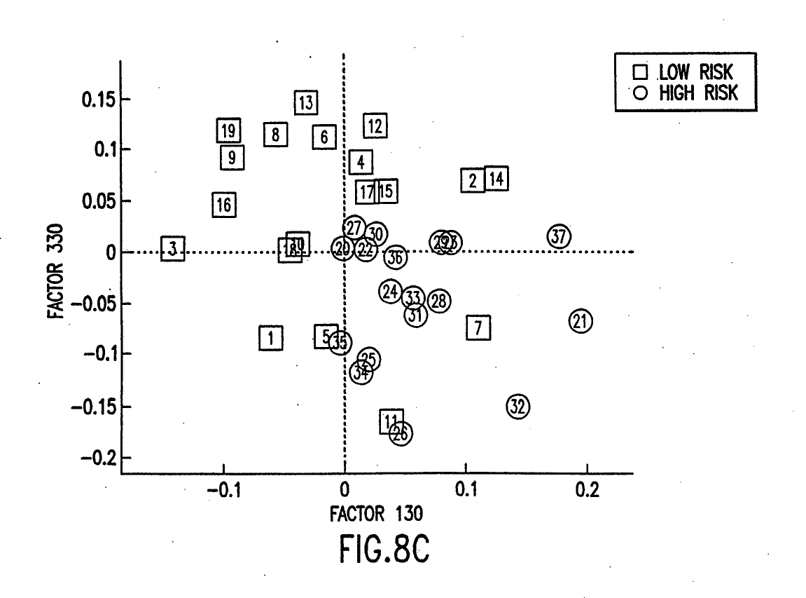

A critical aspect of the analyses described here is allowing the complexity of distinct gene expression patterns to enter the predictive model. Tumors are graphed against metagene levels for three of the highest scoring metagene factors (FIG. 8). This analysis highlights the need to analyze multiple aspects of gene expression patterns. For example, if the low-risk cases 1, 3 and 11 are assessed against metagene 146 alone, their levels are more consistent with high-risk cases. However, when additional dimensions are considered, the picture changes. The second frame (upper right) shows that low-risk is consistent with low levels of metagene 130 or high levels of metagene 146; hence, cases 1 and 3 are not inconsistent in the overall pattern, though case 11 is consistent. An analysis that selects one set of genes, summarized here as one metagene, as a “predictor” would be potentially misleading, as it ignores the broader picture of multiple interlocked genomic patterns that together characterize a state. In the predictions, these two metagenes play key roles: low levels of metagene 146 coupled with higher levels of metagene 130 are strongly predictive of high-risk cases. Metagene 330 also plays a role and it is the combined use of multiple metagenes, in the context of the tree selection model building process that ultimately yields a pattern that has the capacity to accurately predict the clinical outcome.

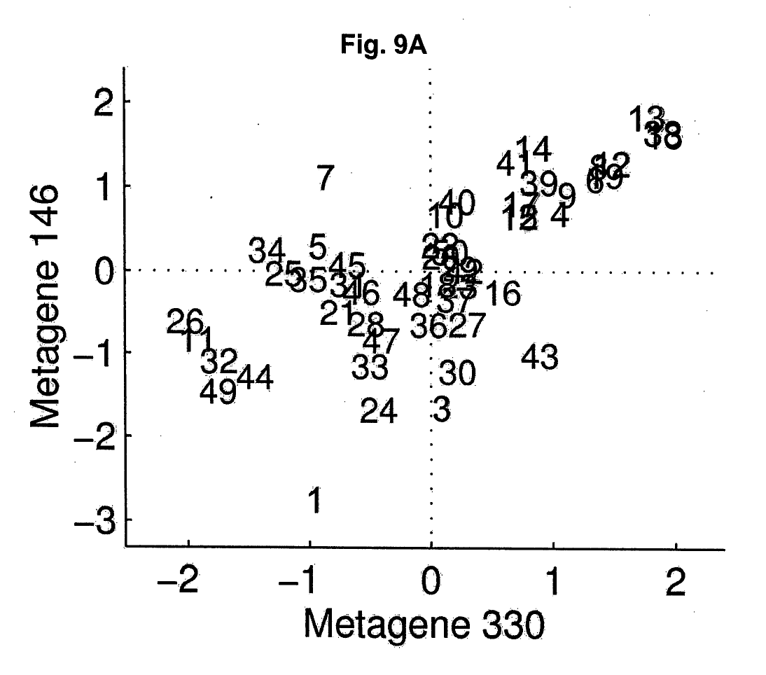

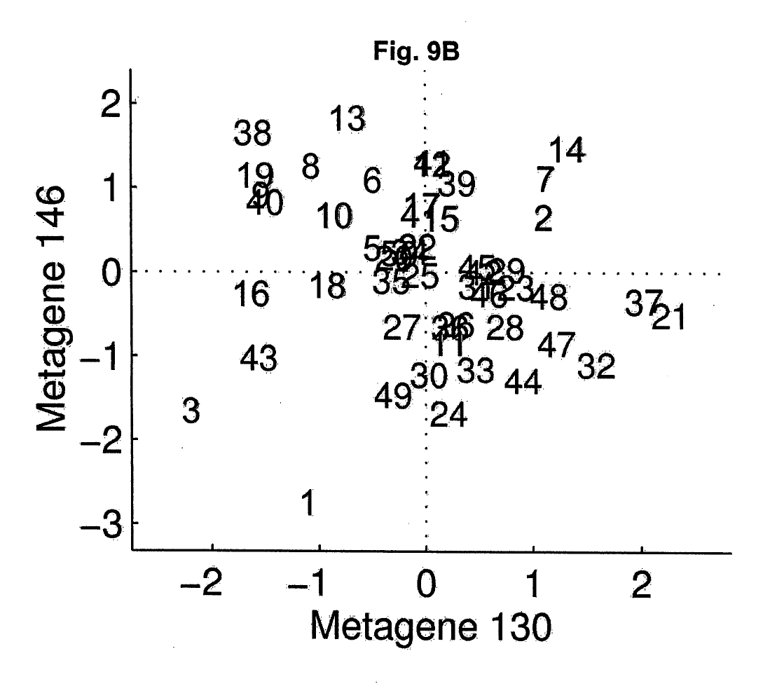

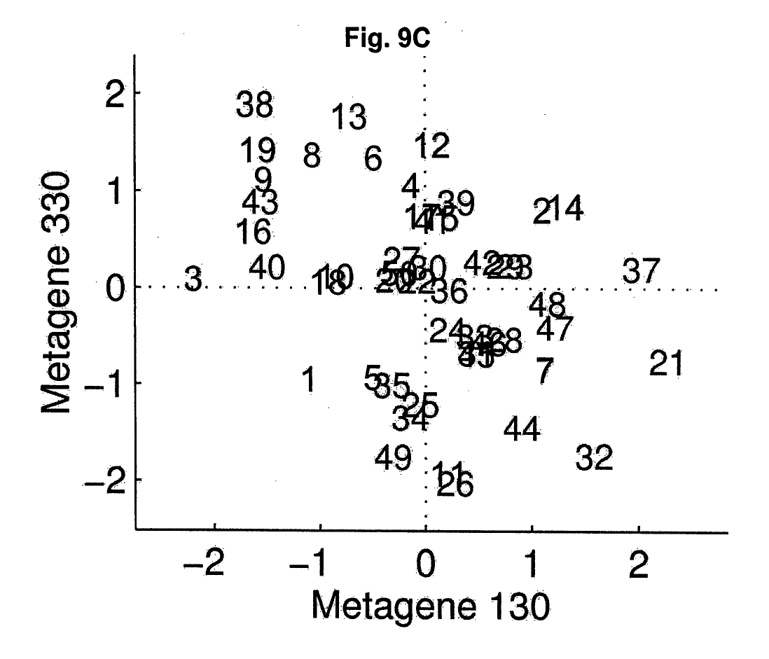

This analysis was validated using data from a study conducted in a prior study. To extend this analysis to an independent data set, we used a small but relevant subset of the patient samples studied in a previous Duke breast cancer analysis (West et al., Predicting the Clinical Status of Human Breast Cancer by Using Gene Expression Profiles, Proc. Natl. Acad. Sci., USA 2001; 98:11462, hereinafter called the “Duke PNAS 2001 Study”). This is a limited initial study conducted using binary regression analysis, but also supportive of the basic conclusion of predictive value of multiple metagene patterns. Relative to the samples used in this analysis which were based entirely on an East Asian cohort, and thus racially homogeneous, the Duke PNAS 2001 study patients had rather different characteristics: the racial difference, and the facts that the US women were generally much older and had much larger tumors at surgery than East Asian women. Furthermore, the numbers of extreme (>9) lymph nodes are very small, so the criteria for the two risk groups were relaxed (ignoring age, reducing the number of positive nodes for the high-risk group, and substantially increasing the maximum tumor size for the low-risk group) in order to generate meaningful numbers of cases for study. This led to 6 low-risk cases (lymph node negative, ER+, tumor sizes less than 3.5 cm which is the median size of the whole group) and 7 high-risk cases (at least 4 positive nodes, rather than 10). Additional complications are due to the fact that the expression data for this older study were obtained on an earlier Affymetrix microarray, so they represent different though overlapping genes. In spite of these complications, and the resulting expectation that predictive accuracy would be reduced, the predictions based on precisely the model fitted to the Asian data are very accurate: one of the low-risks cases appears more consistent, in terms of metagene expression, with the high-risk cases, whereas the remaining 12 cases are very accurately predicted to lie within their defined risk groups. Interestingly, the apparently discrepant low-risk case (#42) has the largest tumor (3.5 cm) of the group. FIG. 9 exhibits the three key metagenes, in a format similar to FIG. 8 but now including also these external validation cases, where concordance with the Asian samples is clear.

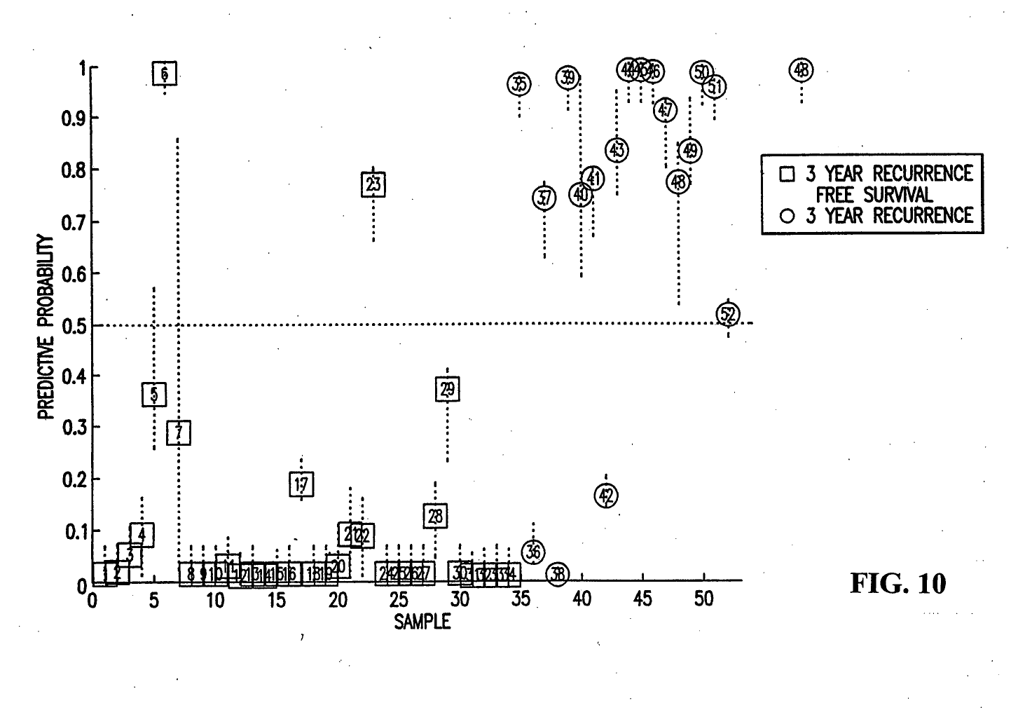

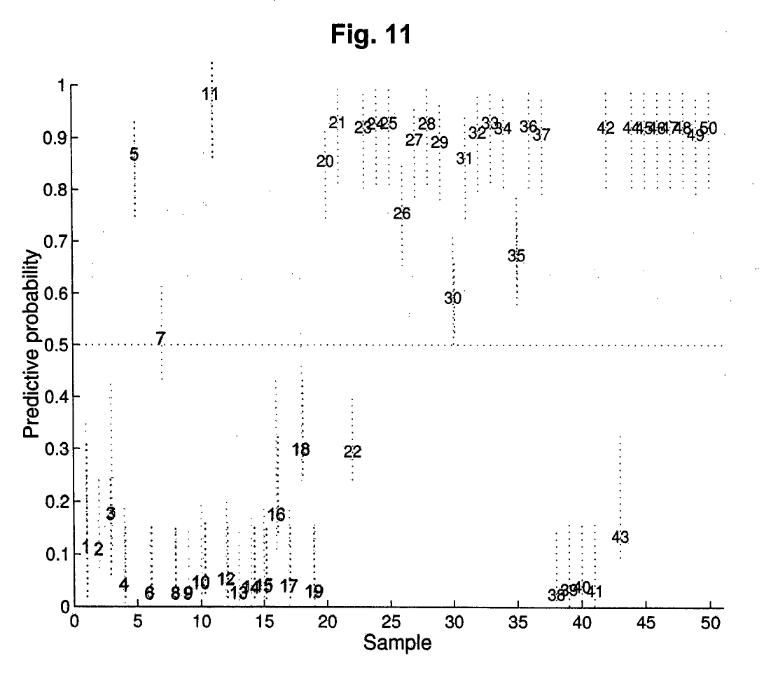

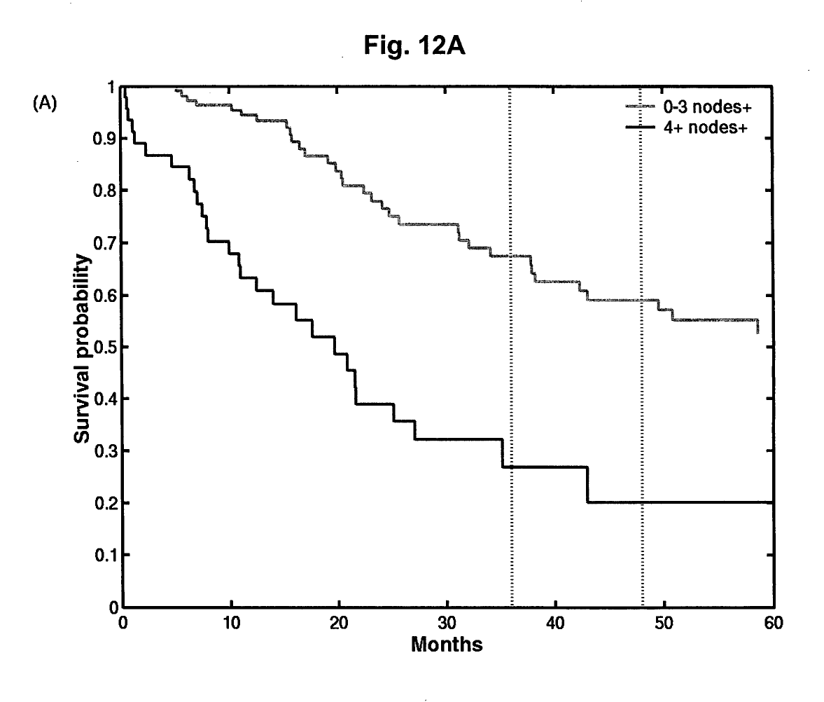

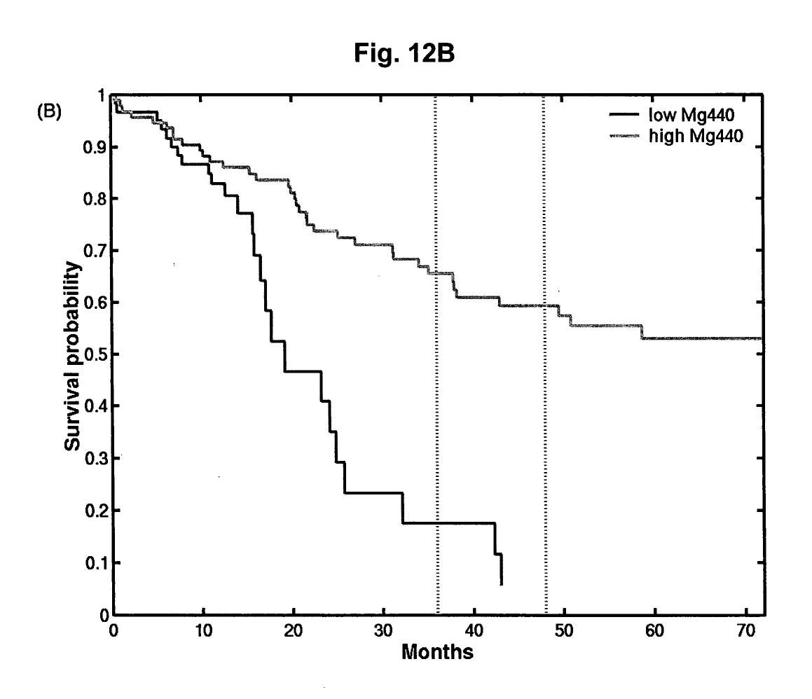

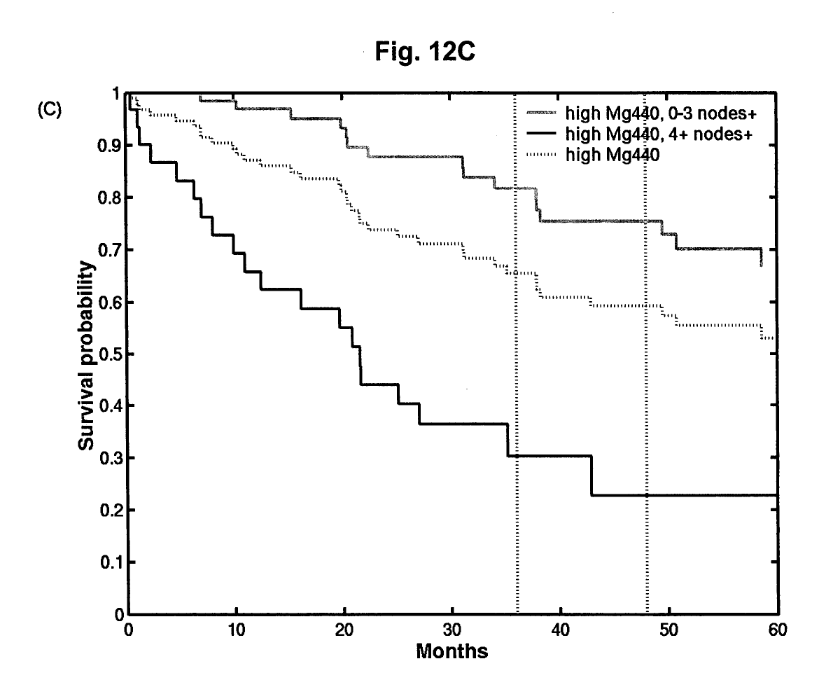

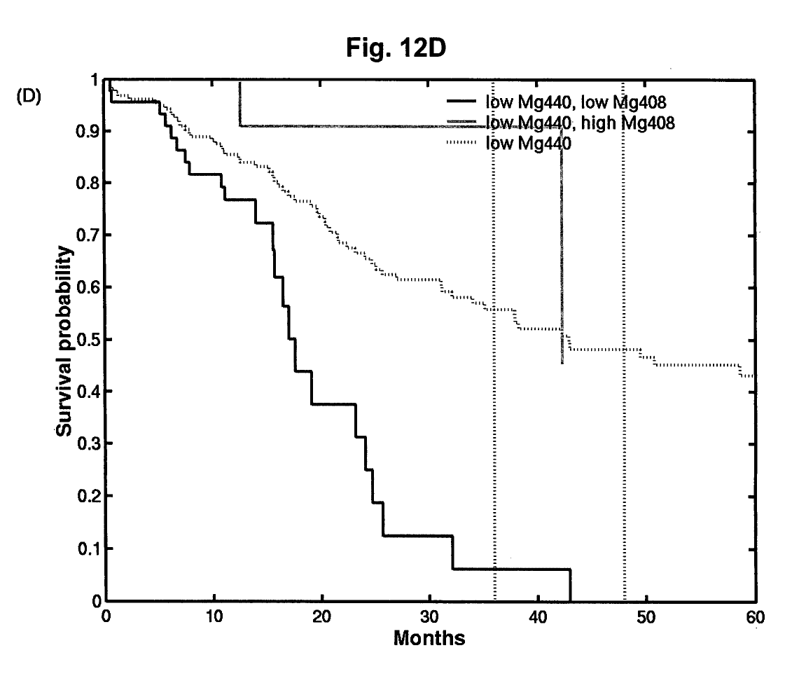

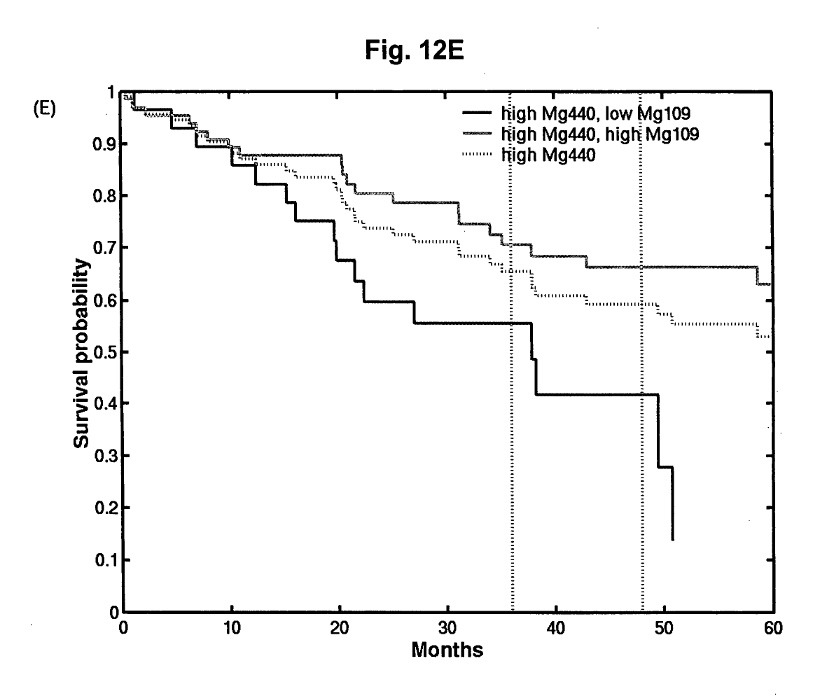

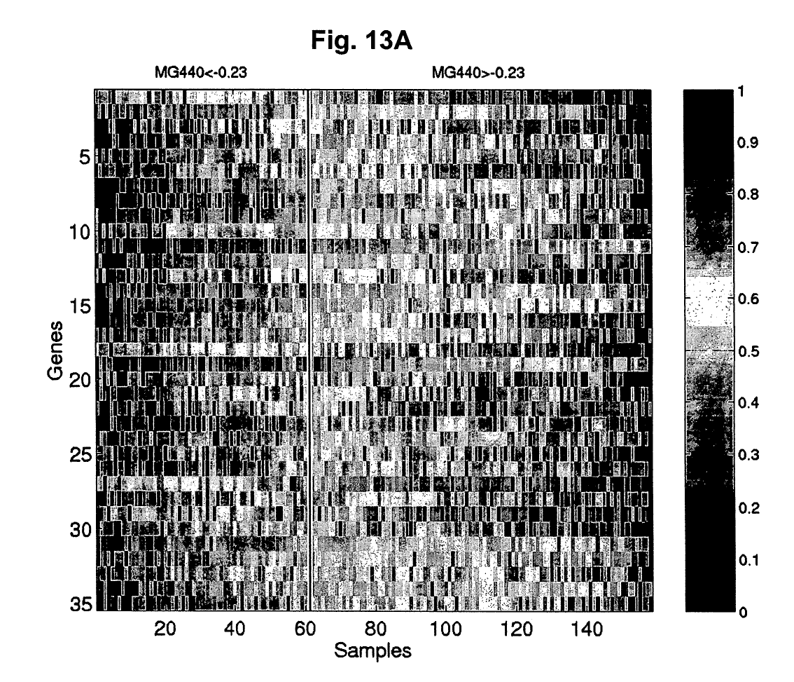

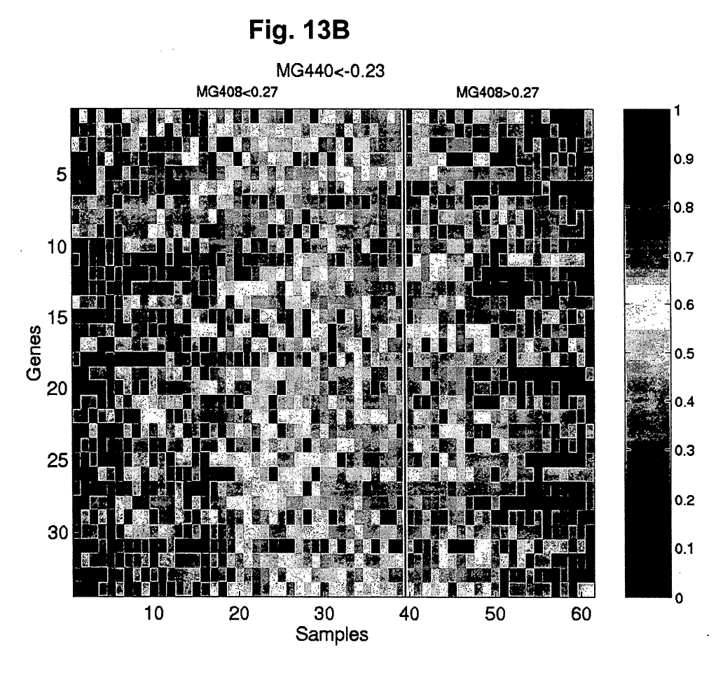

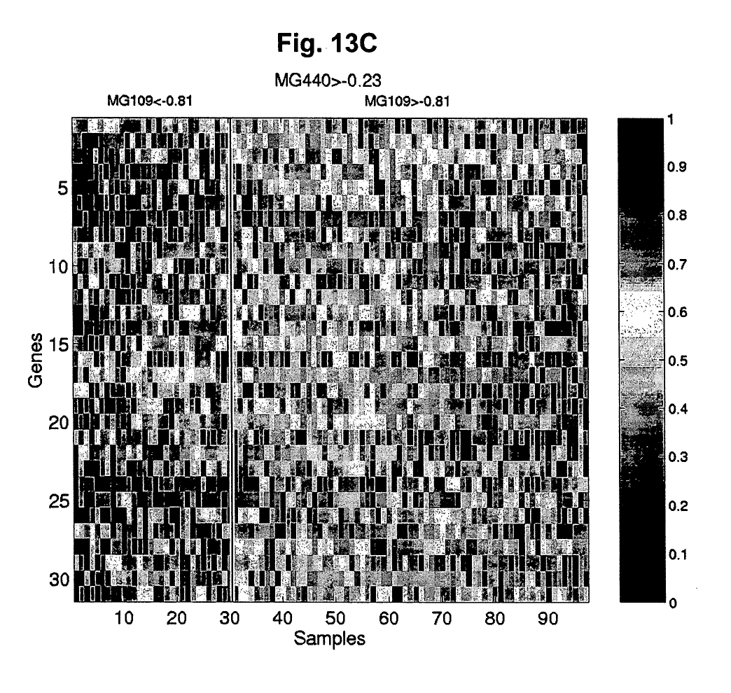

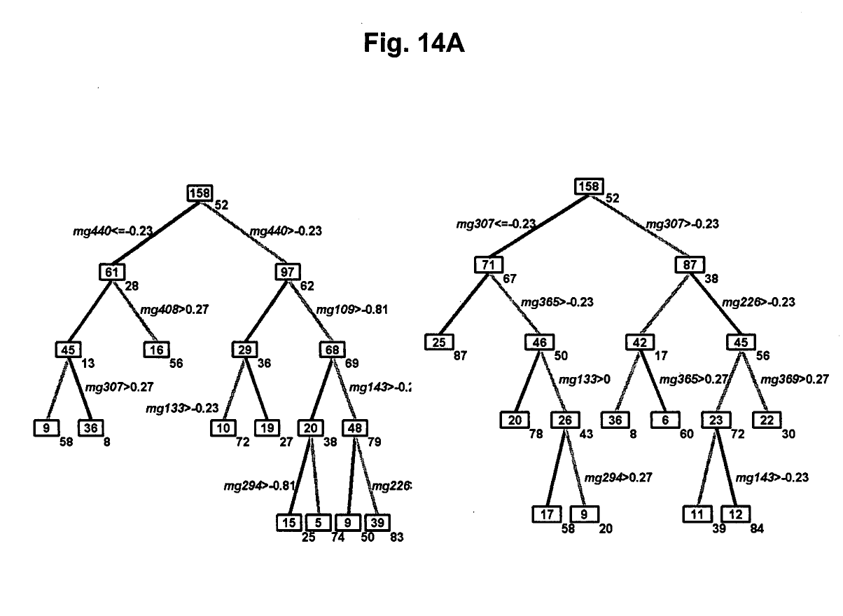

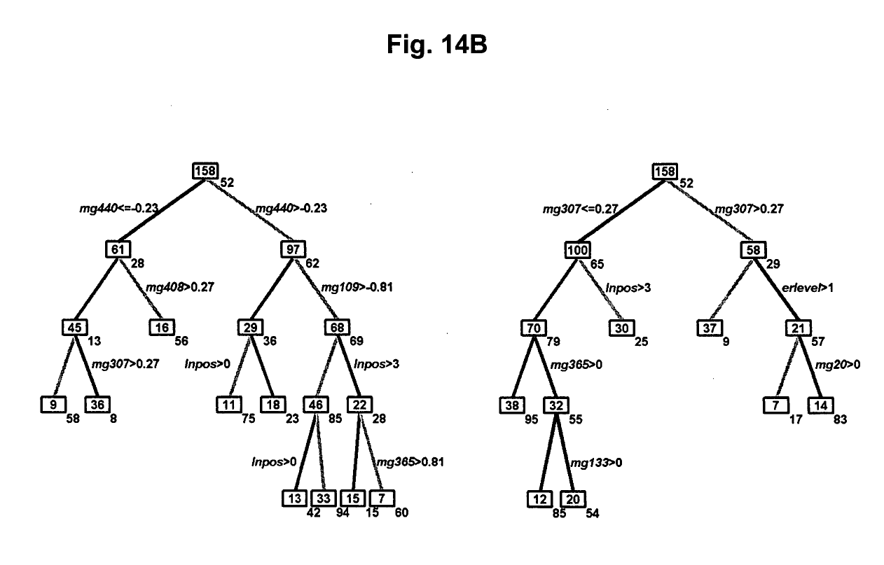

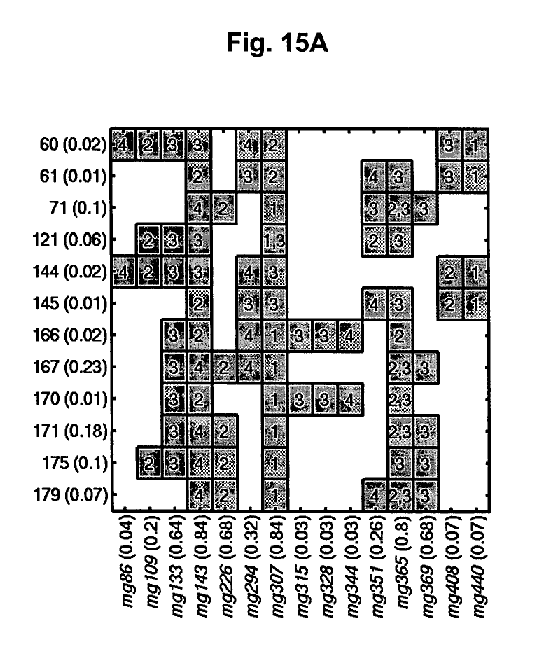

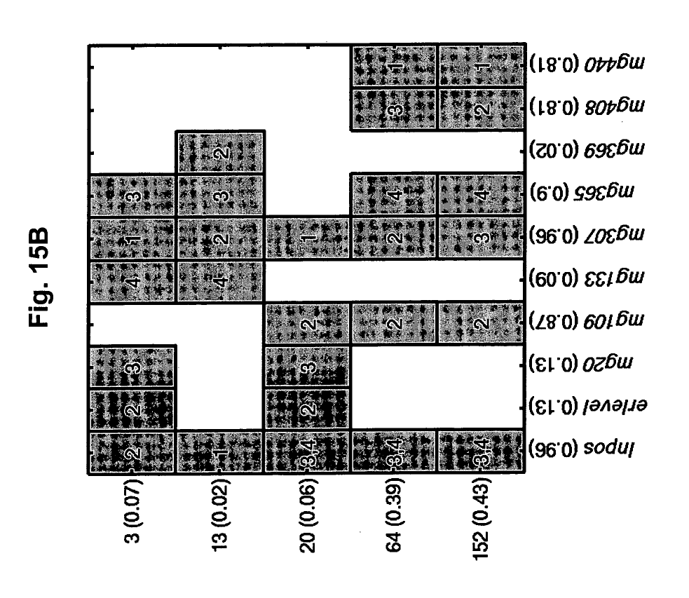

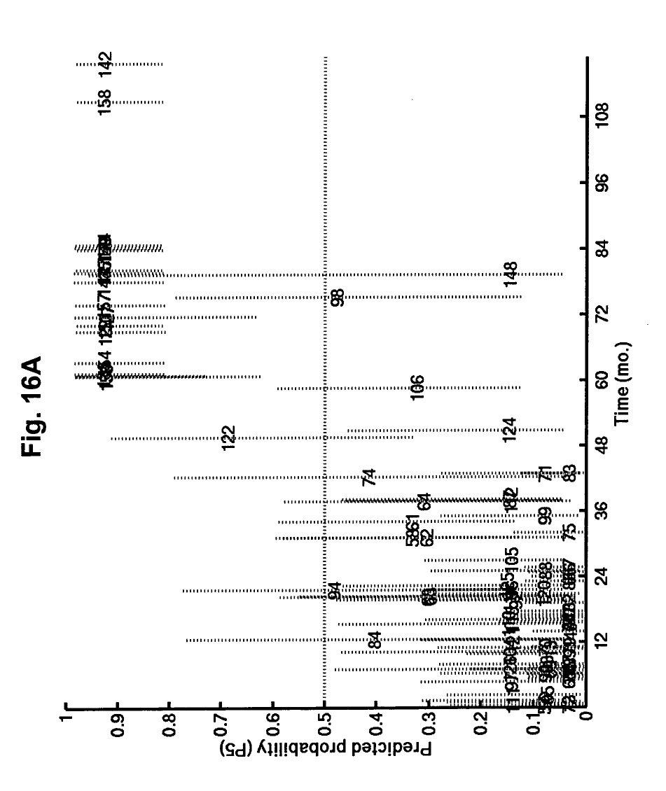

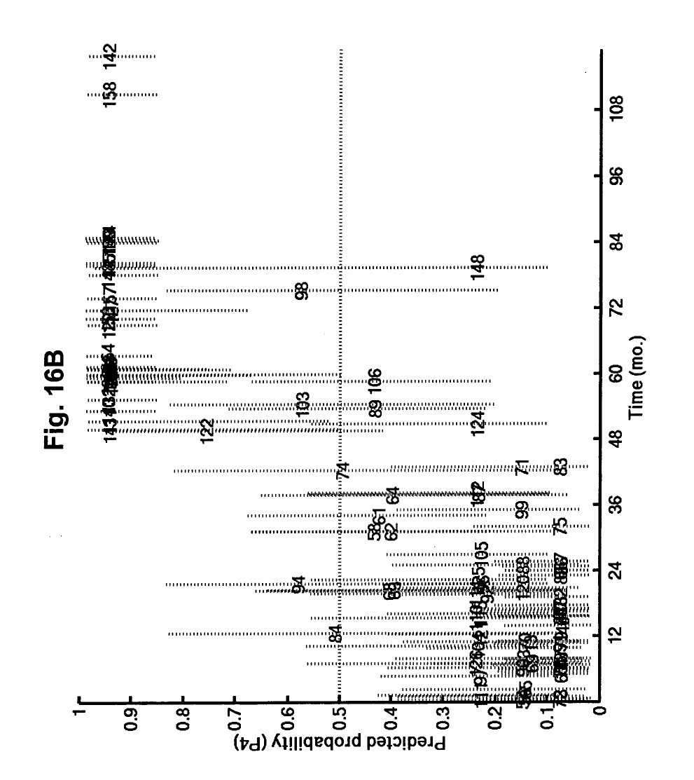

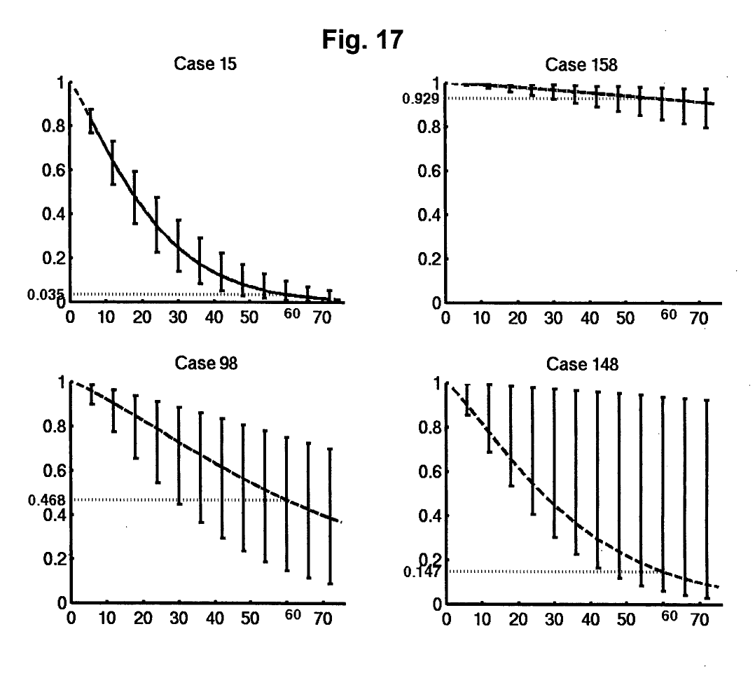

The second analysis concerns 3 year recurrence following primary surgery among the challenging and varied subset of patients with 1-3 positive lymph nodes. Such patients typically receive adjuvant chemotherapy alone, and uniformly across this risk group, so that it is of interest to explain variations in outcome within this subgroup based on predictors other than treatment regimen. This is a critical subgroup as more than 20% suffer relapse within five years (See Cheng et al., Unique Features of Breast Cancer in Taiwan, Breast Cancer Res. Treat. 2000:63:213-23). Hence, improved prognosis for this heterogeneous group is of critical importance; patients identified with a high probability of relapse could be targeted for more intensive treatment. The data set used in this analysis provides expression profiles on 52 cases in this lymph node category (34 non-recurrent, 18 recurrent). The aggregate predictions from the sets of generated statistical tree models defines a rather accurate picture; once again, there is an approximate 90% (with 95% CI 82-99%) overall predictive accuracy in the 52 separate one-at-a-time, cross-validation prediction assessments (FIG. 10).

Based on the gene expression analysis, the 3 year non-recurrent cases 6 and 23, having profiles more akin to recurrent cases, would be candidates for intensive treatment. These patients did receive adjuvant chemotherapy based on additional clinical risk factors (especially tumor size). Thus traditional clinical risk factors other than lymph node status also indicate higher risk of recurrence for these two cases, consistent with the molecular predictions. Each actually survived recurrence-free for over three years; case 6 recurred at 42 months and case 23 remains disease-free after over 6 years. Cases with low genomic criteria for recurrence would be 36, 38 and 42. They, however, each recurred within three years. These are cases that, under prognois informed by only the genomic model, would have been indicated as more benign and not candidates for intensive treatment, whereas such a treatment might have proven to be more beneficial.

Genes Implicated in Lymph Node and Recurrence Studies

Subsets of genes related to the metagene predictors of lymph node involvement are replete with those involved in cellular immunity including a high proportion of genes that function in the interferon pathway. Genes associated with metagene predictors of lymph node metastasis are provided in Table 3. Genes associated with metagene predictors of breast cancer recurrence are provided in Table 4. A Full list of genes defining all metagenes is shown in Table 6. Table 6 is provided at the end of the specification for the purpose of convenience.

They include genes that are induced by interferon such as various chemokines and chemokine receptors (Rantes, CXCL10, CCR2), other interferon-induced genes (IF130, IF135, IFI27, IF144, IFIT1, IFIT4, IFITM3), as well as interferon effectors (2′-5′ oligoA synthetase), and genes encoding proteins mediating the induction of these genes in response to interferon (STAT1 and IRF1). This connection is intriguing given the role of interferon as a mediator of the anti-tumor response and, together with the fact that many genes involved in T cell function (TCRA, CD3D, IL2R, MHC) are also included within the group that predict lymph node metastasis. Possibly, this may reflect the distinct nature of these tumors that have acquired a metastatic potential that elicits an anti-tumor response that is ultimately unsuccessful or an aberration of the normal anti-tumor response. Both of the key metagenes, 146 and 330, contain a number of these interferon related genes.

There is little intersection between the lists of genes defined by key metagenes here and those from the Duke 2001 PNAS lymph node study, which is perhaps not surprising given the relative heterogeneity of the patients in the Duke study. However, when the method of analysis used previously is reapplied to the restricted subset of 6 low versus 7 high risk cases identified in the external validation study reported above, the 100 genes that most strongly relate to the categorization of lymph node status do indeed overlap with the top few metagenes of the current study. In particular, these include several genes already noted that are involved in an interferon response (STAT1, MX1, IFIT1, ISG115, IF127, and IFI44).

Genes implicated in recurrence prediction do not exhibit such a striking functional clustering but do include many examples previously associated with breast cancer. Moreover, this group of genes is clearly distinct set from those that predict lymph node involvement. They include genes associated with cell proliferation control, both cell cycle specific activities (CDKN2D, Cyclin F, E2F4, DNA primase, DNA ligase), more general cell growth and signaling activities (MK2, JAK3, MAPK8IP, and EF1α), and a number of growth factor receptors and G-protein coupled receptors, some of which have been shown to facilitate breast tumor growth (EpoR). Possibly, the poor prognosis with respect to survival reflects a more vigorous proliferative capacity of the tumor.

We conclude that genes implicated in the prediction of lymph node metastasis and overall recurrence of disease, although clearly representing interrelated phenomena, nevertheless reflect the participation of distinct biological processes. The modeling approach we take here is flexible in this regard. The tree models select only those metagenes that are most relevant to the prediction in hand and also enable a more accurate analysis.

The instant invention by allowing the integration of clinical and genomic factors, allows for personalized medicine that aims to characterize those variables unique to the individual that determine disease susceptibility, response to therapy, and eventual disease outcome. It does so by addressing this in assessing complex, multivariate patterns in gene expression data from primary tumor biopsies, and in exploring the value of such patterns in predicting lymph node metastasis and relapse. The resulting predictive accuracy of about 90%, and additional understanding of individual outcomes generated by the analysis, confirm the utility of gene expression patterns as prognostic factors in breast cancer. The invention stresses the focus on predictions made in terms of numerical probabilities of outcomes for individual patients, with associated measures of uncertainties.

The lymph node risk group analysis defines metagene patterns capable of predicting high versus low risk cases with good accuracy, in both internal and external validation studies. In a reanalysis of the small subset of samples from the Duke 2001 PNAS Study that relate most closely to the risk categories defined in this current study, it is determined that improved predictions relative to earlier methods were seen, but also that a number of genes, including interferon-induced genes and others, were in common. This provides additional support for the biological relevance of the metagene predictors identified, and suggests potential areas for further pathway studies. In one embodiment, the present invention would allow for the prediction of drug metabolism pathways that occur in a individual patient. The concordance between genomic predictors found between the Asian and US samples, though preliminary, is also a positive finding.

A related recurrence study (T. Van Veer et al., Gene Expression Profiling Predicts Clinical Outcome of Breast Cancer, Nature, 2002, 4154:530-6) defines a single summary of gene expression related to breast cancer recurrence (though not nodal metastasis), generating a 70 gene predictor. The methods of the instant invention do not identify more than 17 of these 70 genes on the Affymetrix array used here, and none of these appears in the key metagenes in the recurrence study. The analysis approach used in T. Van Veer et al follows the work of the Duke 2001 PNAS Study in developing a single predictor based on an initial screen for genes most correlated with outcome. However, a major distinction of the current invention relative to these prior studies is the finding that multiple measures of gene expression—multiple metagenes—may be found that are involved in explaining differences and, most importantly, defining predictions. Investigation of several metagenes, defining distinct patterns in the data relevant to the outcome, show how the combined effect of several views of clinico-biological data can highlight the similarities between patients while also identifying their differences. The non-linear statistical analysis aids in the elucidation of such patterns as they shed light on individual cases, as well as providing for informed predictions based on multiple patterns.

This latter point relates to the broader question of utilizing gene expression profiles into prognostic settings. The present invention allows for the integration of genomic data with clinical risk factors that will determine the strategy for treating patients as individuals with distinct genomic disease features. Although, genomic data may not replace traditional clinical risk factors, it will add significant detail to this clinical information, especially in a context such as breast cancer where multiple, interacting biological and environmental processes define physiological states, and individual dimensions provide only partial information. As one initial example, the recurrence study here focuses on the 1-3 positive lymph node group where the analysis defines metagenes optimized for prediction within that group; predicting other subgroups, such as higher-risk cases in terms of lymph node count or subgroups stratified by additional clinical factors, will involve exploration of metagenes that optimally relate to outcomes within those subgroups.

Reliably improved predictions of disease course, including lymph node metastasis or recurrence, will profoundly affect the clinical decision process. Several studies indicate that 22-33% of node negative tumors behave in a manner similar to node positive tumors (Polychemotherapy for Early Breast Cancer: An overview of the randomized trials, Early Breast Cancer Trialists Collaborative Group, Lancet 2001: 352:930-42). Whether an issue of timing or of the inability to recognize histopathalogic involvement of tumor material in the lymph nodes, a capacity to identify these cases as requiring more intensive clinical intervention could lead to an improvement in cancer survival. Previous attempts to correlate characteristics of primary tumors such as S-phase fraction, tumor grade, ploidy, c-erbB-2 overexpression, and hormone receptor status with lymph node metastasis have proven unsuccessful (See Mittra I, MacRae K D. A Meta-analysis of reported correlations between prognostic factors in breast cancer: does axillary lymph node metastasis represent biology or chronology, Eur. J. Cancer 1991;27:1574-83; McGuire W L. Prognostic factors for recurrence and survival in human breast cancer. Breast Cancer Res Treat. 1987; 10:5-9; Tandon A K, Clark G M, Chamness G C, Ullrich A, McGuire W L. HER-2/neu oncogene protein and prognosis in breast cancer. J. Clin. Oncol. 1989; 7:1120-8). The ability to appropriately utilize gene expression profiles provides opportunity to add enormous additional detail to the few, currently used biological attributes in tumor characterization. Finally, genes implicated in these analyses generate information of value for future pathway studies, with the potential to identify new targets that may feed into improved therapeutic strategies as well as improved understanding of genes related to the biology of metastasis and tumor evolution.

TABLE 1
Clinical characteristics f patients in the study

| | Number | Percentage |
|---|---|---|
| Age | | |
| <40 | 27 | 30.3 |
| 41-50 | 26 | 29.2 |
| 51-60 | 19 | 21.4 |
| >60 | 17 | 19.1 |
| Histology type | | |
| Infiltrating Ductal Carcinoma | 78 | 87.6 |
| Infiltrating Lobular Carcinoma | 2 | 2.3 |
| Papillary Carcinoma | 2 | 2.3 |
| Tubular Carcinoma | 1 | 1.1 |
| Cribriform Carcinoma | 1 | 1.1 |
| Apocrine Carcinoma | 1 | 1.1 |
| Others (mixed of histologies) | 4 | 4.5 |
| Pathological tumor size | | |
| <1 cm | 6 | 6.8 |
| 1-2 cm | 31 | 34.8 |
| 2-5 cm | 47 | 52.8 |
| >5 cm | 5 | 5.6 |
| Lymph node positive | | |
| 0 | 19 | 21.4 |
| 1-3 | 52 | 58.4 |
| 4-9 | 0 | 0 |
| >10 | 18 | 20.2 |
| Nuclear grade | | |
| Grade I | 15 | 16.8 |
| Grade II | 24 | 27.0 |
| Grade III | 50 | 56.2 |
| LVI (peritumoral and intratumoral) | | |
| Absent | 35 | 39.3 |
| Focal | 16 | 18.0 |
| Prominent | 38 | 42.7 |
| ER status | | |
| Positive | 74 | 83.1 |
| Negative | 15 | 16.9 |

TABLE 2
Clinical information on discordant cases

| | | | | | | | | | |
|---|---|---|---|---|---|---|---|---|---|
| | | | | | Tumor | | | | |
| Case # | Surgery | RT | CT | Histology | size | Nodes | ER | PR | Relapse |

CM

| | | | | | | | | | |
|---|---|---|---|---|---|---|---|---|---|
| LN-5 | MRM | N | F | IDC | 2 | 0 | +++ | ++ | NED, 12 months |
| LN-7 | MRM | N | No | IDC | 1.7 | 0 | +++ | +++ | Yes, 32 months |
| LN-11 | BCS | Y | No | IDC | 0.5 | 0 | + | +++ | Yes, 38 months |
| LN-22 | MRM | Y | CEF | IDC | 3 | 10 | + | + | Yes, 75 months |
| Rec-38 | MRM | N | No | TC | 1.8 | 2 | + | ++ | Yes, 11 months |
| Rec-23 | MRM | N | CAF | IDC | 3 | 1 | − | − | NED, 74 months |
| Rec-6 | MRM | N | CMF | ILC | 3.1 | 2 | + | + | Yes, 44 months |
| Rec-36 | MRM | N | No | IDC | 3.5 | 1 | + | − | Yes, 6 months |
| Rec-42 | MRM | N | CEF | IDC | 3 | 2 | + | + | Yes, 16 months |

Abbreviations:
MRM, modified radical mastectomy;
RT, adjuvant Radiotherapy;
CT, adjuvant chemotherapy;
BCS, breast conserving surgery;
NED, no evidence of disease;
IDC, infiltrating ductal carcinoma;
ILC, infiltrating lobular carcinoma;
TC, tubular carcinoma.

Table 3: Genes associated with metagene predictors of lymph node metastasis See end of disclosure.

Table 4: Genes associated with Metagene Predictors of Breast Cancer Recurrence See end of disclosure.

Table 5: Full List of Genes Defining All 496 Metagenes as Determined in Example 3A (See End of Disclosure)

### EXAMPLE 3B

### Prediction of Outcomes in Individual Breast Cancer Patients

(i) Combining Multiple Metagene Signatures to Improve the Accuracy of Breast Cancer Recurrence Prediction

The analyses employing the method of the invention utilizes the data from 158 breast cancer patients registered at the Koo Foundation Sun Yat-Sen Cancer Center (KF-SYSCC) in Taipei during 1991-2001 (See Chen, S. H. et al. Unique features of breast cancer in Taiwan. Breast Cancer Res Treat. 63, 213-223 (2000)), with detailed clinical records of traditional risk factors—axillary lymph node status, ER status, age, tumor size, nuclear grade, recurrence, and others (See Table 1). Gene expression assays provide data summarized in terms of multiple metagenes (See Huant, E. et al. Gene expression predictors of breast cancer outcomes. Lancet in press, (2003); Seo, D. M. et al.).

Samples used, extract preparation, and labeling. The case study involved 158 primary tumor biopsies at the Koo Foundation Sun Yat-Sen Cancer Center (KF-SYSCC) in Taipei, collected and banked between 1991-2001. Samples were collected under Duke (IRB# 3157-01) and KF-SYSCC (Sep. 21, 2001) Institutional Review Board guidelines. Total RNA was extracted from tumor tissue with Qiagen RNEasy kits, and assessed for quality with an Agilent Lab-on-a-Chip 2100 Bioanalyzer. Hybridization targets (probes for hybridization) were prepared from total RNA according to standard Affymetrix protocols.

Hybridization procedures and parameters. The amount of starting total RNA for each reaction was 20 μg. Briefly, first strand cDNA synthesis was generated using a T7-linked oligo-dT primer, followed by second strand synthesis. An in vitro transcription reaction was performed to generate the cRNA containing biotinylated UTP and CTP, which was subsequently chemically fragmented at 95° C. for 35 min. The fragmented, biotinylated cRNA was hybridized in MES buffer (2-[N-morpholino]ethansulfonic acid) containing 0.5 mg/ml acetylated bovine serum albumin to Affymetrix GeneChip Human U95Av2 arrays at 45° C. for 16 hr, according to the Affymetrix protocol (www.affymetrix.com and Pittman Ms-NG 21 www.affymetrix.com/products/arrays/specific/hgu95.affx). The arrays contain over 12,000 genes and ESTs. Arrays were washed and stained with streptavidin-phycoerythrin (SAPE, Molecular Probes). Signal amplification was performed using a biotinylated antistreptavidin antibody (Vector Laboratories, Burlingame, Calif.) at 3 μg/ml. This was followed by a second staining with SAPE. Normal goat IgG (2 mg/ml) was used as a blocking agent. Each sample was hybridized once.

Measurement data and specifications. Scans were performed with an Affymetrix GeneChip scanner and the expression value for each gene was calculated using the Affymetrix Microarray Analysis Suite (v5.0), computing the expression intensities in ‘signal’ units defined by software. Scaling factors were determined for each hybridization based on an arbitrary target intensity of 500. Scans were rejected if the scaling factor exceeded a factor of 25, resulting in only one reject. Files containing the computed single intensity value for each probe cell on the arrays (CEL files), files containing experimental and sample information (control info files), and files providing the signal intensity values for each probe set, as derived from the Affymetrix Microarray Analysis Suite (v5.0) software (pivot files), can be found in the Supplementary Material on the project web site.

Array design. All assays employed the Affymetrix Human U95Av2 GeneChip. The characteristics of the array are detailed on the Affymetrix web site (www.affymetrix.com/products/arrays/specific/hgu95.affk).

Statistical analysis. Statistical analysis of the gene expression data involves a number of approaches. Initial exploratory analyses of clinical and genomic patterns associated with recurrence are based on traditional Kaplan-Meier and proportional hazards models. The core methodology that underlies our comprehensive clinico-genomic models uses statistical prediction tree models, and the gene expression data enters into these models in the form of what we term metagenes. As previously described, metagenes represent the aggregate patterns of variation of subsets of potentially related genes. Our current approach is to cluster genes with similar patterns of expression and evaluate a single underlying “signature” of each cluster; this signature is termed a metagene for that cluster and serves as a candidate predictive factor in statistical models. Complete technical details of the clustering analysis methods, the construction of metagene summaries, and the development and implementation of statistical analysis via predictive classification tree models, are given in the accompanying Supplementary Material.

Survival curve estimation using Kaplan-Meier estimates and Cox proportional hazards models illustrates the traditional view of stratifying patients into high versus low risk of recurrence based on clinical factors such as lymph node involvement (See FIG. 12A). Similar survival rate summaries using any one of a number of metagenes indicate stronger association with recurrence. Metagene 440 (Mg440) provides a strongly discriminating genomic signature (See FIG. 12B): individuals in the “low Mg440” group exhibit a raw 3-year survival rate of about 20%, compared to about 65% in the “high Mg440” group. This is similar to a recent study described in the previous section employing a single 70-gene predictor that classified breast cancer patients into risk categories based on a “good” or “poor” signature. However, although the prediction of low-risk (good signature) was accurate, the prediction of high-risk (poor signature) was highly uncertain since individuals in this group had a 50-50 probability of recurrence at 10 years (See van de Vijver, M. J. et al. A gene-expression signature as a predictor of survival in breast cancer. N. Engl. J. Med. 347, 1999-2009 (2002). The Mg440 predictor alone is more accurate, in this sense, at the shorter (and more challenging) 3-year horizon, but this analysis only begins the process of understanding personal-level recurrence risks. Further factors are available to substantially refine these risk categories towards customized, personal prediction and to generate improved understanding of uncertainties for the individual patient.

An examination of the gene expression pattern defined by the Mg440 split (See FIG. 13) reveals substantial heterogeneity in the patterns in the two subgroups. Considering that additional gene expression patterns might resolve this heterogeneity, metagenes were examined for further, statistically significant categorization. As a result, the “low Mg440” group splits further on Mg408, while the “high Mg440” group splits on Mg109 (See FIG. 13). In each case, the expression patterns were further divided into more homogeneous subgroups based on the expression patterns of a second metagene.

The value of this refinement is clear in the Kaplan-Meier estimate in which the incorporation of additional metagenes markedly changes the survival estimates (See FIGS. 12D & 12E). This combination of multiple metagenes via further categorization of patients into refined risk groups underlies our statistical tree models and leads to substantially improved predictions—suggested by the figure. The same applies to combining clinical factors with metagenes (See FIG. 12C). Also, multiple metagenes are capable of playing significant roles in such analyses (See Tables 2 and 4). Thus, it is clear that there is a resulting potential for different models to generate different, even potentially conflicting predictions. Understanding this is vital in developing an appreciation of the true nature of the genomic state, reflected in multiple, related measures of expression. Hence there is a need to consider multiple models that define successive partitions of patient groups with a mechanism to formally compare, contrast and combine them.

(ii) Statistical Tree Models Utilizing Multiple Metagenes to Predict Cancer recurrence

To explore multiple metagenes for optimal predictions, the invention uses extensions of regression and classification trees determined by the statistical model. A single tree defines successive partitions of the sample into more homogenous subgroups. At any node of the tree, the corresponding subset of patients may be divided into two at a threshold on a chosen metagene, analogous to the standard low/high-risk grouping already discussed. The analysis shown in FIG. 13 represents one node of a tree in which Mg440 splits the samples into two groups that are then further split by additional metagenes. The logical extension is to tree models with more levels, and also to multiple trees. At any node, the optimal metagene/threshold pair for dividing the sample in the node is chosen by screening all metagenes, and evaluated by a test statistic for the significance of splits across a range of possible thresholds. A split is made if the significance exceeds a specified level. Tree growth is restricted, and ended, when no metagene can be found to define a significant split. Multiple possible splits generate copies of the tree and so underlie the generation of forests of trees. The specific statistical test used is a Bayes' factor (integrated likelihood ratio) test (See Kass, R. E. & Raftery, A. E. Bayes' factors. J. Am. Stat. Assoc. 90, 773-795 (1998)) that is generally conservative relative to standard significance tests and so tends to generate less elaborate trees than traditional tree programs.

Two highly significant tree models, involving several metagenes are shown in FIG. 14A, where the development of branches involving additional metagenes, and the resulting predictions of recurrence within the population subgroups are defined by each leaf. The boxes at nodes of a tree indicate the number of patients together with the model-based estimate of 4-year recurrence-free survival probability. These simple point estimates of recurrence probabilities help to illustrate the implications of the tree model; as a patient is successively categorized down the tree, these node probabilities show the “current” prediction at each node and how those predictions change as additional predictor variables are used. It must be borne in mind, of course, that these point estimates are subject to uncertainty generated by the analyses (see FIGS. 16 and 17). For example, the 50% probability indicated in the extreme left-hand terminal node of the first tree in frame (A) is in fact very uncertain, with associated confidence intervals spanning up to much higher values well above 90%.

At any given node of a tree model, there may be several metagenes defining significant subgroups, so it is important to consider multiple tree models. A resulting set of tree models is evaluated statistically by computing the implied value of the statistical likelihood function for each tree; the set of likelihood values are then converted to tree probabilities by summing and normalizing with respect to all selected trees. Predictions are based on all trees in combination, via weighted averages of predictions from individual trees with the tree probabilities acting as weights. This “model averaging” is well known to generally improve prediction accuracy relative to choosing one “best” model (See Hoeting, J., Madigan, D., Raftery, A. E. & Volinsky, C. T. Bayesian model averaging. Statistical Science in press, (1999); Clyde, M. Bayesian Statistics 6. Bernardo, J. M. (ed.), pp. 157-185 (Oxford University Press, 1999)) especially when several or many models fit the data comparably. In exploring and evaluating trees, several hundreds are generated and weighted; very low probability trees are discarded and the remaining are summarized and averaged to compute resulting predictions.

(iii) Statistical Prediction Tree Models Combining Metagenes and Clinical Risk Factors Predict Individual Breast Recurrence Most Accurately

The tree models were extended to explore all forms of input data, both genomic and clinical. Key clinical factors are lymph node status, represented as 0, 1-3,4-9, and 10 or more positive nodes, ER status (0,1,2+), tumor size, and treatment factors. FIG. 3B displays two of the most highly significant trees that play important roles in contributing to the prediction of recurrence. The key clinical variable identified by these trees is nodal status; its appearance in these most highly weighted trees indicates that it supersedes some of the metagene predictors selected in the exclusively genomic analysis. ER status defines secondary aspects of some of the top trees. Of hundreds of trees generated in the model search, others involve clinical predictors and also treatment variables, but these trees receive low relative statistical likelihood measures and resulting tree probabilities. Treatment protocols follow closely the traditional clinical risk groups that are dominated by lymph node status, and so, though some lesser weighted trees involve variants of treatments in appropriate ways, the inclusion of nodal status stands-in for treatments in highly weighted trees.

Once lymph node status is a candidate predictor, it defines key aspects of predictive trees and reduces the number of metagenes required to achieve accurate predictions. ER status (ER level) is the second clinical factor selected in some of the top trees, and appears here in conjunction with Mg20 that in fact defines a group of genes related to the known risk factor Her-2-nu/Erb-b2. One minor feature (lowest level, right branch) of the first tree is worth noting—a final split according to node negatives versus nodes 1-3 positive. This represents a partition of this subgroup into the traditional two lowest lymph node risk categories, but associates higher risk with the subgroup of node negatives in this final branch of this path in the tree. The reason is twofold: first, the sample design over represented short-term recurrences among the lymph node negatives, second, the 1-3 lymph node positives tend to have some form of adjuvant chemotherapy so are treated more aggressively. The model isolates these subgroups and identifies the differential risk related to this specific aspect of sample selection for this data set, though this feature would be refined in further analysis of a larger, more balanced sample.

FIG. 15A summarizes the tree model-predictor variable for the most highly weighted trees based solely on metagenes; FIG. 15B summarizes that using both metagenes and clinical factors. These represent subsets of hundreds of trees that were evaluated, and account for most of the resulting predictive value. The figures indicate the predictor variables (columns) that appear in the selected top trees (rows), and the levels (boxed numbers) of the trees in which they define node splits. The probability of each tree and the overall probability of occurrence of each of the clinical and metagene factors across the set of trees are also given. Metagenes dominate the initial splits. Other tree models—with lesser relative weights but nevertheless representing interesting combinations of predictor variables—include additional metagenes that are strongly related to those in the top few trees. Although each of the two models (metagenes only versus combined metagenes and clinical factors) defines significant models and are substantially accurate in cross-validated prediction assessments, the combined models have a significantly higher statistical likelihood (difference in log-model likelihoods is greater than 11, which represents a very substantial weight of evidence in favor of the clinico-genomic model).

(iv) Predicting Risk of Recurrence Based on Tree Model Summaries

Honest assessment of true predictive accuracy of the models can be made based on a one at-a-time cross-validation study in which the analysis is repeatedly performed—for example, holding out one tumor sample at each reanalysis and predicting the recurrence time distribution for that holdout patient. Importantly, the entire model building process—selection of metagenes and clinical factors, and their combination in sets of trees to be weighted by the data analysis—must form part of each reanalysis in order to obtain a truly honest predictive evaluation. No pre-selection of predictor variables, or pre-specification of aspects of the model, may be made based on an examination of all the data prior to these repeat validation analyses, as such would bias the results towards what will generally be a gross overstatement of predictive accuracy and validity.

FIG. 16 displays summaries of this honest predictive assessment for 5-year survival probabilities (panel A) and 4-year survival probabilities (panel B). Corresponding to the point estimates, receiver-operator characteristic (ROC) curves were computed that indicate the capacity to predict 4-year survivors with over 90% accuracy, and 5-year survivors with about 95% accuracy. That is, by simply classifying a patient as “high-risk” versus “low-risk” based on her predicted recurrence probability, about 90% (or 95%) of cases are correctly predicted in the sense of low-risk cases not recurring and high-risk cases recurring. Although this is a very crude summary of overall prediction accuracy a more detailed analysis is available in the next example. Nevertheless, serves to indicate a very high degree of model accuracy. Consistent with the fitted model, the combined clinico-genomic analysis exceeds the predictive accuracy of the exclusively genomic analysis. In addition to providing predictive evaluation, this provides an initial illustration of the use of such models in individual patient-level predictions.

Although a number of patients with shorter follow-up do not appear in the figures, because their status as 4- or 5-year survivors is undetermined the models directly predict their survival distributions and provide assessment of survival chances conditional on the observed time of recurrence-free follow-up (See FIG. 18) again at the individual level.

(v) Metagenes can Predict and Substitute for Clinical Risk Factors

The combined clinico-genomic predictive tree analyses reveal that lymph node involvement appears in the key predictive trees, consistent with the wide recognition of lymph node involvement as the most significant clinical risk factor in breast cancer (See Jatoi, I., Hilsenbeck, S. G., Clark, G. M. & Osborne, C. K. Significance of axillary lymph node metastasis in primary breast cancer. J Clin Oncol 17, 2334-2340 (1999); McGuire, W. L. Prognostic factors for recurrence and survival in human breast cancer. Breast Cancer Res Treat. 10, 5-9 (1987)). Since axillary node dissection carries significant morbidity, the invention uses a metagene analysis as a preferable alternative to clinical lymph node diagnosis. The metagene signatures have the capacity to replace nodal counts although the latter still aids in constructing the most significant models. Nevertheless, when tree analyses are carried out without the use of clinical factors, including lymph node status, the predictive capability is very good indeed, almost comparable to the combined model though still overshadowed to a degree, in terms of statistical fit and predictive accuracy.

Metagene 408 is a key feature of one major “branch” of the most significant trees (See FIG. 14A, the left branch of trees beginning with Mg440). The association of Mg408 as a strong predictor of lymph node status (see, Huang, E. et al. Gene expression predictors of breast cancer outcomes. Lancet in press, (2003)) indicates that it can, to some degree, substitute for lymph node status subject to verification and comparison by the model of the invention. In the model with genomic data alone, the picture is less clear as many more metagenes are required to define a larger set of relatively equally well weighted trees, representing multiple patterns that each partially substitute for the clinical predictors. Among these is Mg328, an additional genomic predictor of lymph node status.

Also included are Mg315 and Mg351 that correlate with genes within the estrogen pathway substitute for ER status in the genomic-only analysis. See Example 2.

A further case, Mg20 that appears with ER status in the combined model, is based on 15 genes that define the Her-2-neu/Erb-b2 metagene cluster (See Table 4). Her-2-neu/Erb-b2 has previously been defined as a risk factor primarily among ER negative cases (see, Tandon, A. K., Clark, G. M., Chamness, G. C., Ullrich, A. & McGuire, W. L. HER-2/neu oncogene protein and prognosis in breast cancer. J. Clin. Oncol. 7, 1120-1128 (1989)) so its appearance here within a subset of ER positive cases implicates Her-2-nu/Erb-b2 more broadly. Its strength as a prognostic factor is, however, only marginal and it is strongly dominated by preceding metagenes.

(vi) Prediction of Recurrence to Achieve Personalized Prognosis

The 4- and 5-year survival probability predictions in FIG. 16 are taken from the full survival distributions that result from the statistical model analysis. At each terminal leaf of each tree, the analysis estimates a full survival time distribution that represents the survival characteristics of individuals assigned to the subpopulation with predictors defining that leaf. Formal predictions for an individual are based on averaging these survival distributions across tree models, each tree weighted by its corresponding data-based probability. The analysis also provides assessments of uncertainty about predicted survival curves; communicating these uncertainties along with estimates is critical to interpretation and assessment of survival prospects at an individual level. To illustrate this, FIG. 17 displays the resulting predictions for four patients whose clinical and metagene factors match a chosen four of the patients in the data base. Each panel gives the predicted survival curve for one patient. At a number of time points, the vertical intervals represent approximate 95% uncertainty intervals for the predicted survival probabilities at those time points. Also, the estimated 5-year survival probability is highlighted.

A critical aspect of predictive analysis is that models must properly evaluate uncertainties associated with predictions of probabilities of recurrence and other outcomes. Uncertainties arise from multiple sources, including the usual sampling variability and the limitations of samples sizes. Uncertainty also arises when the patient characteristics that define predictions show evidence of conflict. The tree model framework utilizes multiple trees and, in cases of apparent conflict within or between the genomic and clinical predictor sets, different trees may suggest different outcomes. It is then important that an overall prediction summary recognizes and represents this via high uncertainty intervals about probability predictions, and that the model be open to investigation so that the specifics of such cases can be explored.

Cases 15 and 158 are examples in which the confidence of prediction, whether for early recurrence (Case #15) or disease-free survival (Case #158), is very high—indicated by the narrow prediction intervals. In contrast, the two additional cases are examples where uncertainty is high. For example, Patient #98 is a younger woman with 10 positive nodes and a reasonably large tumor at biopsy. She was, by choice, not treated aggressively, but in spite of her high clinical risk profile survived recurrence-free up to 75 months. The model predictions clearly indicated substantial conflict among the metagene-clinical predictors, resulting in a very uncertain predictive distribution. A second patient, #148, is an older woman who had one positive node and only a modest sized tumor, so was apparently clinically low-risk and indeed survived recurrence free for at least 6.5 years. The prediction for this individual from the full model was quite uncertain, favoring higher-risk but generating very wide intervals and so suggesting caution and further detailed investigation at the point of evaluation. In fact, the pathology reports for this woman indicated a range of characteristics that defined her as very high-risk (4B by T-staging-15), in contrast to the generally, but not exclusively, lower-risk clinical factors. Further detailed investigations revealed that, in fact, the clinical determinations were highly unusual, with evidence of an invasive, more aggressive tumor, to the extent that the clinical classification of this patient is also, alone, quite controversial. However, the metagene predictors are capable of capturing a very high degree of conflicting information in genomic patterns, perfectly consistent with this very unusual, and complex, mix of conflicting clinical and pathological characteristics. Although the clinico-genomic model dominates the metagene-only model overall, the predictions for Patient #148 in the latter, while similarly uncertain, generate higher point estimates of survival probabilities, and so represent, postfacto, a more accurate prediction for this one individual.

Patient #148 is unusual. Other patients with low (0-3) positive lymph node counts are similarly predicted with low recurrence-free survival probabilities, but much less uncertainty, and in fact recur within four or five years. These cases, and others in the low lymph node count categories that in fact survived much longer, are all very accurately predicted based on the amalgam of risk factors represented in the model.

The analysis framework has the capacity to evaluate the relative contributions of multiple forms of data, both clinical and genomic, to predict disease outcomes. This provides a mechanism to substantially refine predictions to be specific for individual patients. Multiple, related patterns of gene expression—metagene signatures—provide strong and predictively valid associations with breast cancer recurrence. Several key metagenes are each individually capable of defining very highly significant population differences, and their value as population risk factors far exceeds that of previously published genomic risk factors. When combined in predictive models, small sets of multiple metagenes together define improved predictions via successive stratification of the patient set into smaller, more homogeneous subgroups with associated survival distributions defined by interactions of metagenes.

Prediction accuracy can be improved by combining clinical factors with the genomic data. Key metagenes can, to a degree, replace traditional risk factors in terms of individual association with recurrence, but the combination of metagenes and clinical factors, notably axillary lymph node status, defines models most predictive of recurrence. The resulting tree models provide an integrated clinico-genomic analysis that is most highly supported by the data analysis and also generate substantially accurate, crossvalidated predictions at the individual patient level.

The models deliver formal predictive survival assessments, in terms of estimates of survival distributions for future patients, and current patients being followed-up, together with measures of uncertainty about the predictions. The latter are critical in advising clinical decisions. A point prediction of a survival probability, such as a 5-year recurrence probability, is only part of the story; it is critical to also communicate how uncertain that probability estimate is, as measured by an interval estimate that integrates uncertainty due to sample size and sampling fluctuations together with uncertainty arising from potentially conflicting predictors. The specific approach using tree models highlights the latter issue, helping to identify individual patients for whom there is evidence of conflict among the predictors, within or between the genomic and clinical predictors, that is reflected in increased uncertainty about the resulting recurrence predictions.

Genomic data, particularly gene expression profiles, clearly has the capacity to significantly improve clinical predictions. Further, genomic information potentially identifies relevant genes and pathways providing clues to the pathophysiology underlying the disease. Key metagenes that provide predictive power also define sets of genes suggestive of biologically relevant pathways associated with clinical phenotypes. Most striking are the lymph node metagenes, especially Mg408, that involve genes generally associated with tumor immunosurveillance. This indicates that characteristics of the tumor that predict lymph node metastasis, and ultimately disease recurrence as we have shown, relate to the involvement of processes associated with immunological response to the tumor. Immunologically, this may represent an incomplete or failed immunological response, one that allows tumor cells to escape. Alternatively, the immunological response itself may contribute to tumor progression by contributing to local tissue breakdown. Other metagenes highly weighted in predicting disease recurrence, such as Mg440, identify growth-signaling pathways that are altered in a variety of oncogenic settings. Highly related metagenes that have similar weights and contributions to the tree prediction models, such as Mg440 and Mg307, also exhibit similarities in gene function; for example, Mg307 exhibits additional genes associated with growth factor signaling. In contrast, other implicated metagenes identify distinct biological properties suggesting that different aspects of biology are contributing to the prediction and ultimately reflecting the heterogeneity of the disease process. The identification of multiple genes of potential biological relevance to tumor development in breast cancer, and their predictive value in individual-level prognostics models, represents a key and distinctive finding.

In complex diseases such as breast cancer, clinical endpoints reflect the accumulative or aggregate action of multiple genomic patterns—representing multiple gene pathways and their interactions. Individual prognosis must recognize and evaluate such patterns in combination with clinical factors, especially when multiple factors involve conflicting prognostic signals. The invention evaluates and uses multiple, related genomic patterns in combination with clinical factors, rather than a single genomic pattern to the exclusion of other informative factors. Thus, the invention teaches that not only do that multiple factors define the most accurate predictions, also permit the analysis of what may be deemed to be conflicting biological predictors at the clinical evaluation stage.

The modeling process provides a framework in which other forms of clinical data including, but not limited to improvements in clinical phenotyping, new forms of genomic data (for example, DNA structure, protein patterns, metabolic profiles, single nucleotide polymorphisms [SNPs] and haplotype data could be incorporated that will likely make significant contributions to the ultimate prediction of outcome. The generation of predictive models that can evaluate multiple, distinct forms of data thus has the added advantage of being able to integrate any form of quantifiable information. This adaptability is immediately relevant in the context of developing extended studies that aim to refine and evolve the understanding of multiple forms of data relevant to moving genomic analysis through clinical trials to clinical practice.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1. A classification tree model incorporating Bayesian analysis for the statistical prediction of binary outcomes.

2. The tree model of claim 1, wherein the prediction of a binary outcome is dependent on the interaction of data comprising at least two predictor variables.

3. The tree model of claim 2, wherein the data arises by case control design such that the number of 0/1 values in the response data is fixed by design.

4. The tree model of claim 3, such that the case control design assesses association between predictors and binary outcome with nodes of a tree.

5. The tree model of claim 4, such that the Bayesian analysis comprises using sequences of Bayes factor based tests of association to rank and select predictors that define a node split.

6. The tree model of claim 5, further comprising the forward generation of at least one class of trees with high marginal likelihood, wherein the prediction of said class of trees is conducted using principles of model averaging.

7. The tree model of claim 6, wherein the principle of model averaging comprises the steps of:
weighted prediction of a tree by determining its implied posterior probability by a score;
evaluation of the score to exclude unlikely trees;
evaluation of the posterior and predictive distribution at each node and leaf of a tree; and
application of said posterior and predictive distribution to the evaluation o of each tree and the averaging of predictions across trees for future predictive cases.

8. The tree model of claim 1 or 2, wherein the binary outcome is a clinical state.

9. The tree model of claim 1 or 2, wherein the binary outcome is a physiological state.

10. The tree model of claim 1 or 2, wherein the binary outcome is a physical state.

11. The tree model of claim 1 or 2, wherein the binary outcome is a disease state.

12. The tree model of claim 1 or 2, wherein the binary outcome is a risk group.

13. The tree model of claim 1 or 2, wherein the data is biological data.

14. The tree model of claim 1 or 2, wherein the data is statistical data.
